(19) United States
(12) Patent Application Publication
(10) Pub. No.: US 2023/0331781 A1
(43) Pub. Date: Oct. 19, 2023

(54) PAR4 DERIVED PEPTIDES, ANALOGS AND USES THEREOF

(71) Applicants: Yissum Research Development Company of the Hebrew University of Jerusalem Ltd., Jerusalem (IL); Hadasit Medical Research Services and Development Ltd., Jerusalem (IL)

(72) Inventors: Rachel BAR-SHAVIT, Shoresh (IL); Chaim GILON, Jerusalem (IL); Amnon HOFFMAN, Jerusalem (IL); Beatrice UZIELY, Mevasseret-Zion (IL)

(21) Appl. No.: 18/323,210

(22) Filed: May 24, 2023

Related U.S. Application Data
(63) Continuation-in-part of application No. 17/431,867, filed on Aug. 18, 2021.
(60) Provisional application No. 17/431,867, filed on Aug. 18, 2021.
(60) Provisional application No. 62/808,325, filed on Feb. 21, 2019.
(60) Provisional application No. 63/345,454, filed on May 25, 2022.

(51) Int. Cl. C07K 7/64; A61P 35/00

(52) U.S. Cl. CPC C07K 7/64; A61P 35/00

(57) ABSTRACT

The present invention provides peptides derived from the cytoplasmic region of protease-activated receptors 4 (PAR₄) as well as analogs and cyclic analogs, such as backbone cyclic analogs, of these peptides. Pharmaceutical compositions comprising said peptides, analog, cyclic analogs and well as conjugates thereof are provided as well. The peptides, analogs and conjugates of the present invention and pharmaceutical composition comprising thereof have several uses including treating cancer such as cancer expressing PAR proteins such as cancer expressing ErbB protein and triple negative cancer. and inhibiting interactions between PARs and protein comprising PH-domain.

### CROSS-REFERENCE TO RELATED APPLICATIONS

This is a Continuation-In-Part of U.S. patent application Ser. No. 17/431,867, filed on Aug. 18, 2021, which is a national phase application of PCT/IL2020/050185, filed on Feb. 19, 2020, which claims priority from U.S. Provisional Patent Application No. 62/808,325, filed on Feb. 21, 2019 and also claims priority to U.S. Provisional Patent Application No. 63/345,454, filed on May 25, 2022, each of which is hereby incorporated by reference in its entirety.

### FIELD OF THE INVENTION

The present invention relates to peptides derived from cytoplasmic region of PAR₄, analogs thereof, compositions comprising said peptides or analogs as well as use thereof in treating cancer.

### SEQUENCE LISTING

The instant application contains a Sequence Listing which has been submitted electronically in xml format and is hereby incorporated by reference in its entirety. Said xml file, created on 19 Jun. 2023, is named HDST-056 US-1 CIP Revised sequence listing 19Jun.23.xml and is 72,070 bytes in size.

### BACKGROUND OF THE INVENTION

Among the protein modules that drive intermolecular interactions in cellular signaling, the pleckstrin homology (PH) domain is most common. PH domains are mainly recognized by their structural characteristics. They are known to be versatile modules in protein-protein and protein-lipid interaction platforms in a plethora of physiological events. PH domain containing proteins represent a wide diverse group of kinases (such as protein kinase B, Akt), guanine exchange factors, structural and docking proteins.

It was previously demonstrated that the pleckstrin-homology (PH) binding motifs within the C-tails of protease-activated receptors 1 and 2 (PAR₁ and PAR₂, respectively), with a dominant role of PAR₂, are crucial for breast cancer development (Jaber et al., Cell Mol Life Sci. 2014, (13):2517-3). This is mediated through the recruitment and association of signal proteins that harbor a PH-domain. PAR species belong to the large G-protein coupled receptor (GPCR) rhodopsin-like class A family, and comprise four members: PAR₁, PAR₂, PAR₃, and PAR₄. The activation of PARs is mediated by proteolytic cleavage of their N-terminal portion and exposure of an internal ligand, specific for each PAR member, binding consequently to extracellular loop 2 for the initiation of cell signaling.

PAR₁ and PAR₂ play a central role in cancer growth and development, allocating a dominant role for PAR₂. WO 2012/090207 described isolated PAR₁ and PAR₂ cytoplasmic tail peptides and their role in inhibition of these PARs' signal transduction and their use in treating cancer. It was shown that PAR₃ functions mainly as a co-receptor. PAR₄, an important receptor for thrombin-induced cellular responses, is often coexpressed with PAR₁. In-fact, thrombin activation of human platelets is carried out by both PAR₁ and PAR₄ (Reya et al., Nature, 2001, 414:105-111). PAR₄ displays a lower affinity for thrombin than PAR₁, and, as an outcome, PAR₄ was initially hypothesized as a “back-up” receptor. However, studies have shown that PAR₁ and PAR₄ play distinct roles in platelet activation. While PAR₄ function appears to be more essential for the later stages, PAR₁ controls the early stages of platelet activation. Indeed, signaling kinetics exhibited by the two receptors support this hypothesis, whereby PAR₁ signaling is rapid and transient in comparison to that of PAR₄, which has a slower start but a prolonged duration. The transcriptional profile of selected GPCR family was analyzed using high-throughput RNA sequencing. The expression of 195 GPCRs was either up- or down-regulated during somatic reprogramming to cancer stem cells (CSCs) and sphere formation of cancer stem cell. Among GPCRs that are significantly upregulated in CSC sphere formation are PAR₂ and PAR₄. Hence, PAR₂ and PAR₄ play a yet unknown role/s in cancer stem cell properties.

Breast cancer is the most common malignant tumor in women and the second leading cause of cancer death among women overall. Breast cancer is divided into Luminal A (ER+/PR+/HER2−), Luminal B (ER+/PR+/HER2+), HER2+(ER−/PR−/HER2+) and Basal-like (ER−/PR−/HER2−), wherein ER is estrogen receptor, PR is progesterone receptor, HER2 is a proto-oncogene. Basal-like breast cancer is also referred to as triple-negative breast cancer (TNBC).

The four different types of breast cancer are not the same in clinical prognosis, and the methods of treatment are different. Luminal A needs internal secretion treatment as maintenance treatment, Luminal B and HER2+ can use some single anti-drug for targeted therapy, triple-negative breast cancer can use only chemotherapy and cannot use hormone-based therapy.

Peptides are favorable candidates as therapeutic agents due to their wide contribution to physiological processes. However, their usually poor drug-like properties and their non-selective activity, mainly their intrinsic low stability to enzymatic degradation and poor oral bioavailability, limit peptides clinical potential (Ovadia et al., Expert Opin Drug Discov. 2010 Jul. 5(7):655-71). Recent developments in the determination and prediction of the three dimensional (3D) structure of peptides have enabled significant progresses in the field. Some of these advances were aimed to overcome the shortcomings of peptides as drugs.

Drug-like properties refer to pharmacokinetic (PK) properties of the molecule: absorption, metabolism, distribution, excretion and toxicity. These affect directly the systemic exposure of the body to an administered drug and its metabolites. In addition, there is a need for enhanced stability in the blood, across the gastrointestinal (GI) tract and to first pass metabolism in the liver as also chemical stability for effective formulation into a stable dosage form. Chemical modifications can affect the physicochemical properties of peptides and thus may have an impact on their pharmacological activities. For example, cyclization of peptides has been shown to improve chemical stability and hence extend the biological half-life compared to their linear counterparts. Cyclized peptides and peptidomimetics integrate the pharmacological features and biological activity necessary for effective research tools and therapeutics. In general, these structures demonstrate a better maintenance of bioactive conformation, cell permeability and stability compared to their linear counterparts, while maintaining support for a diversity of side chain chemistries. Cyclic peptides usually exhibit high biological activities, as well as a better potency and augmented selectivity compared to their linear analogs, making them ideal candidates for therapeutic lead compounds. However, cyclization can hamper the bioactivity of a linear compound if the method compromises their chemistries To overcome restrictions associated with traditional peptide cyclization, two additional methodologies were developed. These methodologies are divided into N-backbone and C-backbone cyclizations and allow for new modes of cyclization in addition to the classical ones that are limited to cyclization through the side chains and/or the amino or carboxyl terminal groups. Backbone cyclization (BC) method was developed (Gilon et al., 1991, BioPolymers, 31, 745-750). BC is a procedure that enables development of cyclic peptides without utilizing the residues that are part of the natural linear peptide, which may be essential for the peptide biological activity, particularly if the peptide is short. The main advantage of this method is that the cyclization linkage is formed between backbone atoms and leaving free atoms of the side chain functional groups, which are classically critical for binding and biological function. In summary, BC utilizes mainly atypical building blocks with an additional linker of customizable length covalently attached to a backbone functional group for the peptide cyclization. This arrangement maintains the regular amino acid functional groups in their bioactive conformation essential to exert biological activity and acquire drug-like properties.

BC was proved to be a valuable tool in methodological conversion of active sites of proteins to cyclic peptides and even to small macrocycles (Hurevich et al., Bioorg Med Chem 2010, 18, (15), 5754-5761; Hayouka et al., Bioorg Med Chem 2010, 18, (23), 8388-8395; Hess et al., J Med Chem 2008, 51, (4), 1026-34). The BC method is used to introduce global constraints to active peptides. It differs from other cyclization methods since it utilizes non-natural building blocks for cycle anchors, mainly N-alkylated amino acids. BC proved superior to other stabilization methods since the resultant peptides had defined structures that led to better selectivity (Gazal et al., J Med Chem 2002, 45, (8), 1665-71; WO 99/65508) and improved pharmacological properties. The use of BC enables a combinatorial approach called “cycloscan”. It was used for generating and screening BC peptide libraries to find lead peptides that overlap with the bioactive conformation (U.S. Pat. No. 6,117,974).

Despite the progress in development of peptides as drugs, there is a shortness of approved drugs based on peptides. There is a clear need for development of additional peptides having drug-like properties for treating various diseases such as cancer. Moreover, considering the shortness of approved drugs for some aggressive types of breast cancer, which are resistant to known therapies, there is a clear need for the development of additional means for such types of cancer.

### SUMMARY OF THE INVENTION

The present invention is based on the unexpected finding that a peptide derived from a pleckstrin homology (PH)-domain binding motif located at the cytoplasmic tail of protease-activated receptor 4 (PAR₄) is capable of inhibiting the interaction between PAR₄ and a protein comprising a PH-domain, Akt (Protein kinase B). This peptide was used to design more active and stable peptide analogs, particularly cyclic peptide analogs. It is demonstrated that the PAR₄ derived peptide and its analogs are capable of inhibiting or preventing signal transduction mediated by PAR₄ via PH-domain binding motif, and therefore can be used in treating diseases mediated by signal transduction involving PAR₄, e.g. cancer. Interestingly it was shown that the the PAR₄ derived peptide and its analogs are capable of inhibiting signal transduction mediated by PAR₂ via PH-domain binding motif. Such dual action may be benificial in treatment of diseases mediated by these proteins.

In one aspect, the present invention provides a peptide comprising an amino acid sequence SZ₁Z₂FRDZ₃, (SEQ ID NO: 1) wherein Z₁ is an amino acid selected from a hydrophobic amino acid, a modified hydrophobic amino acid, glycine, a modified glycine or histidine, Z₂ is a negatively charged amino acid and Z₃ is a positively charged amino acid, wherein said peptide consists of from 7 to 25 amino acids. According to some embodiments, the present invention provides a peptide comprising an amino acid sequence SZ₁Z₂FRDZ₃ (SEQ ID NO: 2), a salt or a cyclic analog thereof, wherein said peptide consists of 7 to 25 amino acids, Z₁ is an amino acid residue selected from alanine (Ala), a modified Ala, glycine (Gly), a and modified Gly; Z₂ is a negatively charged amino acid; and Z₃ is a positively charged amino acid. According to some embodiments, the peptide comprises Z₂ is an amino acid selected from aspartic acid (Asp) and glutamic acid (Glu) and Z₃ is an amino acid selected from lysine (Lys), arginine (Arg) and His. According to other embodiments, the peptide comprises amino acid sequence SZ₁EFRDK (SEQ ID NO: 4). According to some embodiments, the peptide comprises an amino acid sequence X₁X₂SZ₁EFRDKX₃X₄X₅ (SEQ ID NO: 5), wherein X₁ is an amino acid selected from Tyr, Phe and Trp; X₂, X₃ and X₅ are each independently an amino acid selected from Ala, Val, Leu, Ile and Gly; and X₄ is an amino acid selected from Arg and Lys. According to some embodiments, the present invention provides a peptide comprising an amino acid sequence selected from YVSAEFRDKVRA (SEQ ID NO: 6) and YVSGEFRDKVRA (SEQ ID NO: 7). According to further embodiments, the present invention provides salts and analogs of said peptides. According to certain embodiments, the analog is a cyclic analog and/or comprises a cyclization.

According to some embodiments, the present invention provides a peptide analog of the peptide comprising amino acid sequence SEQ ID NO: 1. According to another embodiment, the present invention provides a cyclic analog comprising amino acid sequence SEQ ID NO: 1. According to some embodiments, the present invention provides a cyclic analog comprising amino acid sequence SZ₁Z₂FRDZ₃X₃ (SEQ ID NO: 24), wherein Z₁ and X₃ are each independently an amino acid residue selected from Ala, a modified Ala, Gly and a modified Gly, Z₂ is an amino acid selected from Asp and Glu and Z₃ is an amino acid selected from Lys, Arg and His. According to some embodiments, Z₁ is selected from Ala or Gly. According to some embodiments, the cyclic analog comprises an amino acid sequence selected from SGEFRDKG (SEQ ID NO: 25) and SGDFRDHG (SEQ ID NO: 26). According to some embodiments, the cyclic analog comprises two modified amino acids are N^{α}-ω-functionalized amino acid derivatives. The two modified amino acids are capable of forming a bridge via a backbone cyclization. According to some embodiments, the analog comprises an amino acid sequence SZ₁Z₂FRDZ₃X₃ (SEQ ID NO: 34), wherein Z₁ and X₃ are each independently an N^{α}-ω-functionalized amino acid derivative building unit, Z₂ is a negatively charged amino acid and Z₃ is a positively charged amino acid. According to some embodiments, Z₁ and X₃ are selected from Gly-BU and Ala-BU. According to other embodiments, Z₁ and X₃ are covalently bound via urea group to form a backbone cyclization, thereby the cyclic analogs are backbone cyclic analogs. According to certain embodiments, Z₂ is selected from Asp and Glu and Z₃ is selected from Lys and His. According to some embodiments, Z₁ and X₃ are both Gly building units. According to some embodiments, each of the building units independently comprises a (C2-C6) alkyl or (C3-C5)alkyl. According to one embodiment, the backbone cyclic analog comprises a sequence selected from SZ₁EFRDKX₃ (SEQ ID NO: 30) and SZ₁DFRDHX3 (SEQ ID NO: 31), wherein Z₁ and X₃ are both Gly-BU units, each comprising a (C3-C₆) alky covalently bound via urea group. According to some embodiment, the present invention provides a backbone cyclic analog having the structure as depicted in Formula I, wherein n and m are each independently an integer between 3 and 6. According to some embodiments, m=n=4.

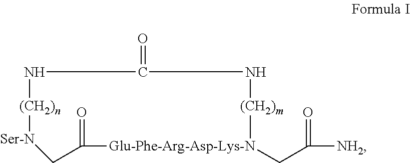

According to some embodiments, the ring size of the cyclic analog is from 29 to 35 atoms. According to other embodiments, the ring of the cyclic analog comprises from 28 to 36 atoms.

According to another aspect, the present invention provides a conjugate of the peptide or cyclic analog of the present invention.

According to another aspect, the present invention provides a pharmaceutical composition comprising a compound selected from the group consisting of peptide, peptide analog, cyclic peptide, cyclic analog, backbone cyclic analog, conjugate and salts thereof, of the present invention, and a pharmaceutically acceptable excipient. According to some embodiments, the pharmaceutical composition is for use in treating a disease mediated by PAR protein. According to one embodiment, the pharmaceutical composition is for use in treating a disease mediated by PAR₄. According to another embodiment, the pharmaceutical composition is for use in treating a disease mediated by PAR₂. According to some embodiments, the pharmaceutical composition is for treating cancer, e.g. for killing cancer stem cells. According to other embodiments, the pharmaceutical composition is for treating carcinoma, e.g. colon cancer or breast cancer.

According to a certain aspect, the present invention provides a method of treating a disease mediated by a protease-activated receptor (PAR) in a subject in need thereof comprising administering a peptide, peptide analog, a conjugate or a pharmaceutical composition comprising said peptide, analog or conjugate of the present invention. According to one embodiment, the PAR is selected from PAR₄ and PAR₂. According to some embodiments, the disease is cancer. In some embodiments, the present invention provides a method of treating cancer expressing EGFR and at least one GPCR selected from PAR₂ and PAR₄ in a subject in need thereof, comprising administering a therapeutically effective amount of a peptide, peptide analog, a conjugate or a pharmaceutical composition comprising said peptide, analog or conjugate as described in any one of the embodiments of the present application.

In a further aspect, the present invention provides a method of inhibiting activation of ErbB protein by at least one GPCR selected from PAR₂ and PAR₄ comprising administering a peptide a peptide, peptide analog, a conjugate or a pharmaceutical composition comprising said peptide, analog or conjugate as described in any one of the embodiments of the present application. In some embodiments, the ErbB is EGFR. In other embodiments, the ErbB is Her2.

According to yet another aspect, the present invention provides a method for inhibiting G-protein coupled receptor (GPCR) mediated signal transduction comprising administering a peptide or an analog thereof capable of selectively inhibiting binding of the GPCR and PH-domain containing protein, wherein said peptide is derived from a PH-domain binding motif of said GPCR. According to one embodiment, the GPCR is PAR₄. According to another embodiment, the GPCR is PAR₂.

According to yet another aspect, the present invention provides a method for inhibiting G-protein coupled receptor (GPCR) mediated signal transduction comprising administering a peptide or an analog thereof capable of selectively inhibiting binding of the GPCR and PH-domain containing protein, wherein said peptide is derived from a cytoplasmic tail (c-tail) of PAR₄ and the GPCR comprises a PH-domain binding motif. According to some embodiments, the GPCR is a PAR. According to one embodiment, PAR is PAR₄ and the protein is selected from Akt, Etk/Bmx and Vav3. According to one embodiment, PAR is PAR₂ and the protein is selected from Akt, Etk/Bmx and Vav3.

According to a further aspect, the present invention provides a method of treating a disease in a subject in need thereof comprising administering a peptide or analog or salt thereof capable of selectively inhibiting binding of a GPCR comprising a PH-domain binding motif and a PH-domain containing protein, wherein said peptide is derived from a cytoplasmic tail (c-tail) of PAR₄. According to some embodiments, the GPCR is PAR. According to one embodiment, PAR is PAR₄. According to another embodiment, the PAR is PAR₂.

### BRIEF DESCRIPTION OF DRAWINGS

FIG. 1 left panel: Immunoprecipitation (IP) analysis between PAR₄ and Akt. HEK 293 cells were transfected with flg-Par4. After 48 hr serum starved cells were AYPGKF activated for PAR₄ and cell lysated were prepared at the indicated time periods. IP were performed using anti flg antibodies (abs). Western blot analysis detected Akt following application of anti Akt. Right panel: Schematic representation of the interaction of PAR₄ protein and with PH-domain of Akt.

FIG. 2A-2B shows the induction of β-catenin stabilization (FIG. 2A) and Lef/Tcf transcriptional activity (FIG. 2B) upon activation of PAR₄, as detailed in Example 1.

FIG. 3A-3D shows the effect of peptide 1 of the interaction of PAR₄ and PH-domain of Akt (FIG. 3A) and on the PAR₄ induced Matrigel invasion (FIG. 3B). Experiments shown in FIG. 3A-3C were carried out in MDA-MB-231 cells. While AYPGKF activation of PAR₄ induces Matrigel invasion of MDA-MB-231 cells, this was attenuated in the presence of CTP4-A. In-contrast, no effect was observed in the presence of peptides CTP4-B or - CTP4-C, similar to non-treated activated parental MDA-MB-231 cells. FIG. 3C shows histograms representing quantification of the cells/HPF invaded the Matrigel layer. Unpaired Student's t test was used. This experiment is a representative of three independent experiments performed in triplicates. FIG. 3D shows that PAR₄ derived peptide CTP4-A potently inhibits PAR₄-Akt association. HEK 293T cells were transfected with wt flg-Par4 followed by AYPGKF activation of PAR₄. IP was performed using anti fig abs of cells treated or not with CTP4-A peptide and immunoblotting with anti-Akt abs.

FIG. 4A-4F shows Akt/PKB associates with PAR₄ via its PH domain. FIG. 4A—PH-Akt module alone binds PAR₄-C-tail but not mutant R25C. HEK 293 cells were transiently transfected with flg-Par4 construct and either with GFP-PH-Akt domain alone or GFP-R25C. IP of cell lysates following PAR₄ AYPGKF activation was carried out using anti-flg abs. Detection of either Akt-PH domain alone associated with PAR₄ was performed with anti-GFP. GFP-PH domain alone were shown to bind PAR₄, no binding was obtained when the mutant R25C of low lipid-binding-affinity was present (FIG. 4B). FIG. 4C—The PI3K inhibitor (Wortmannin) inhibits the binding of Akt to PAR₄. Treatment of HEK 293 cells with LY294002 for various time points or not, following transient transfection with flg-Par4 and AYPGKF activation was performed. Cell lysates were immunoprecipitated with anti-flg antibodies, and anti-Akt was used to assess the association of Akt with the PAR₄ C-tail. A potent inhibition of Akt-PAR₄ association was observed in the presence of Wortmannin FIG. 4D—the F&D amino acids as part of the “FRD” sequence within PAR₄-PH binding domain are essential for PAR₄-Akt association. Amino acid residues of PAR₄- and PAR₂-PH binding motifs. FIG. 4E—mutations inserted into flg-Par4 generated flg-Par4MutE346A. HEK293 cells were transiently transfected with either wt flg-Par4 or flg-Par4MutE346A. IP of cell lysates following PAR₄ activation was carried out using anti-flg abs. Detection of PAR₄-Akt association was performed by anti Akt Abs. While association with wt Par4 is seen after 5 and 10 min AYPGKF PAR₄ activation, when using flg-Par4 E346A no inhibition is seen and effective association with Akt is seen at 30 min activation. FIG. 4F—utations inserted into flg-Par4 generated flg-Par4Mut F347A and flg-Par4Mut D349A. HEK293 cells were transiently transfected with either wt flg-Par4 or flg-Par4Mut F347A or flg-Par4Mut D349A. IP of cell lysates following PAR₄ activation was carried out using anti-f/g abs. Detection of PAR₄-Akt association was performed by anti Akt Abs. While association with wt Par4 is seen after 5 min AYPGKF PAR₄ activation, no such binding is obtained when using either flg-Par4 F347A or flg-Par4D349A.

FIG. 5 shows the effect of cyclic PAR(4-4); Pc (4-4) (desiganated here as cyclic PAR₄ (4×4) inhibitor) on interactions of PAR₄ and Akt.

FIG. 6 shows the effect of cyclic PAR(2-2) inhibitor at two different concentrations: 50 μM and 200 μM on interactions of PAR₄ and Akt.

FIG. 7 shows the effect of cyclic PAR(6-6) inhibitor at two different concentrations: 50 μM and 200 μM on interactions of PAR₄ and Akt.

FIG. 8A-8K shows that PAR₄ c(4-4) inhibits PAR₄-Akt/PKB, Matrigel invasion and migration.

FIG. 8A shows PAR₄-Akt association. HEK293 cells were transiently transfected with flg-Par4 followed by AYPGKF PAR₄ activation in the presence and absence of PAR₄c (4-4) within the range of 50 nM-150 μM. IP of cell lysates following different periods of activation was carried out using anti flg abs. Detection of Akt was performed by anti Akt abs. β-actin serves as a control gene for loading. FIG. 8B shows matrigel invasion of Lovo colon cancer cells. Matrigel invasion before and after AYPGKF PAR₄ activation and in the presence and absence of PAR₄ c(4-4) inhibitor in the activated PAR₄ cells. A range between 50 nM-150 μM PAR₄ c(4-4) inhibitor was applied. While ample Matrigel invasion is seen following PAR₄ activation, this was attenuated in the presence of the inhibitor in all the concentrations used, similar to control prior to PAR₄ activation. FIG. 8C—Histograms represent quantification of the cells/HPF invaded the Matrigel layer. Unpaired Student's t test was used. This experiment is a representative of three independent experiments performed in triplicates. FIG. 8D-8K show Wound-scratch migration assay. Lovo cancer cell-line was grown to confluence. Then, an equal wound scratch was introduced to Lovo cell monolayers. Monolayers were serum starved overnight and PAR₄ was activated or not with AYPGKF. One hour prior to activation PAR₄ c(4-4), either at 150 μM or 300 μM concentrations was added to the monolayers. While the gap in the scratch started to be filled out 24 hr after PAR₄ activation PAR₄ the presence of Pc(4-4) inhibitor, a marked inhibition is observed.

FIG. 9A-9F shows that PAR₄ c(4-4) potently inhibits RKO/Par4 clone/s induced tumor generation. FIG. 9A. A clone of RKO/PAR₄ overexpressing cells (1×10⁶) were injected subcutaneously into nude mice. PAR₄ c(4-4) (100 μM; 0.25 mg/30 gm) was subcutaneously injected, at the site of the tumor either at the same day of RKO/PAR₄ clone inoculation or 4 days later. The inhibitor was applied repeatedly 3 times/week for 3 weeks. Mice were weighted every 3 days and tumor size was measured. Mice were sacrificed after 32 days. FIG. 9B shows tumor volume measurements of RKO/PAR₄ clone inoculated into nude mice. Tumors were weighed and measured for size at the indicated time points and tumor volume was calculated05. FIG. 9C. qPCR of a representative RKO stable clone of PAR₄. Error bars show s.d.; *** P<0.001. FIG. 9D. IHC of RKO/PAR₄ derived tumors. Immunohistochemistry of RKO compared to small tumors in the presence of PAR₄ c(4-4) inhibitor. Levels of proliferation in the large and small tumors were indicated by applying anti ki67 abs (FIG. 9Da and 9Db). For apoptosis, application of active caspase-3 Abs were applied (FIG. 9Dc and 9Dd). While high levels of proliferation is seen in the large tumors (FIG. 9Da) and little in the small tumors (9Db), high levels of active caspase-3 in the small tumors (FIG. 9Dc) were obtained as compared to the very little in the large tumors. FIG. 9E. Histograms representing positive IHC in each treatment group. Error bars show s.d.; * P<0.05; *** P<0.001., FIG. 9F shows H&E staining. Hematoxilyn & eosin staining of large (left panel) and small (right panel) tumors distinctly indicate to the appearance of a wide basement membrane in the small tumors whereas a thinner and cell-invaded basement membrane is observed in large tumors.

FIG. 10A-10B. shows that Pc(4-4) potently inhibits HCT116 induced tumor generation, in vivo.

FIG. 10A shows tumor extracted from mice. HCT116 cells (1×10⁶) were injected subcutaneously to nude mice. Pc(4-4) (100 μM; 0.25 mg/30 gm) was subcutaneously injected, either at the same day of tumor cell inoculation or 4 days later. The inhibitor was applied repeatedly 3 times/week for 3 weeks. Mice were weighted every 3 days and tumor size was measured. Mice were sacrificed after 21 days. This is a representative of three times performed experiments. FIG. 10B shows the measured umor volume. Tumor volume measurements of HCT116 cells inoculated into nude mice. Tumors were weighed and measured for size at the indicated time points and tumor volume was calculated. Error bars show s.d.; * P<0.05; *** P<0.001; while ns are considered as not significant.

FIG. 11 shows the effect of 150 μM cyclic PAR(4-4) inhibitor on interactions of PAR₂ and Akt. HEK293 cells were transiently transfected with Par2 followed by SLIGKV PAR₂ activation in the presence and absence of PAR₄ c(4-4) at 150 μM. IP of cell lysates following different time of activation was carried out using anti PAR₂ abs. Detection of Akt was performed by anti Akt abs. β-actin serves as a control gene for loading.

FIG. 12A-12D. shows that Pc(4-4) inhibits pre-formed RKO/hPar4 tumors. FIG. 12A shows the effect of Pc(4-4) on PAR₄ induced tumors. Tumors generated by the RKO/Par4 clone/s (1×10⁶ cells, subcutaneously). Injections of the inhibitor Pc(4-4) have started 3 weeks post cell inoculation (5 mg/kg, every other day). FIG. 12B—levels of Par4 in stable clone/s of RKO cells. Error bars show s.d.; *** P<0.001. FIG. 12C—pre-formed tumors. Mice bearing tumors formed (˜1 cm) prior to the Pc(4-4) injection. FIG. 12D tumor volume (cm3). Tumors generated throughout the experimental period versus injections of Pc(4-4) initiated 3 weeks post cell inoculation. Each treatment group contained 7 mice. This is a representative of three times performed experiments. Error bars show s.d.; ** P<0.01.

FIG. 13A-13G. shows cross-talk between PAR₄ and EGFR. FIG. 13A—Activation of PAR₄ induces phosphorylation of EGFR. HEK293 cells were co-transfected with 0.4 μg of wt flg-Par4 and 0.8 μg of gfp-egfr. Cells were serum starved over night following AYPGKF PAR₄ activation (200 μM) for 5-45 min. Detection by Western blot analyses of pY-EGFR and EGFR was performed using either anti-phospho tyrosine or anti EGFR antibodies respectively. PAR₄ activation induces EGFR tyrosine phosphorylation within 15 min. FIG. 13B—Pc(4-4) inhibits EGFR phosphorylation. HEK293 cells were co-transfected with 0.8 μg of wt flg-Par4 and 1 μg of gfp-egfr. Cells were serum starved over night following AYPGKF PAR₄ activation (200 μM) for 5-45 min in the presence and absence of PAR₄ c(4-4) (100 μM). Detection by Western blot analyses of pEGFR and EGFR was performed using either anti-phospho tyrosine or anti EGFR antibodies (1:1000 dilution) respectively. PAR₄ activation induces EGFR tyrosine phosphorylation, Pc(4-4), a PAR₄ inhibitor potently inhibited it. FIG. 13C—PAR₄ mutants F347L and D349A abolish pTyr-EGFR. HEK293 cells were transiently co-transfected with either flg-Par4 or flg-Par4D349A or flg-hPar4F347L with egfr1 plasmid. Cells were serum starved over night following AYPGKF PAR₄ activation (200 μM) for 5-45 min. Detection by Western blot analyses of pY-EGFR and EGFR was performed using either anti-phospho tyrosine or anti-EGFR antibodies respectively. In the presence of flg-Par4 potent pTyr-EGFR1 is observed within 15′ and 30′ AYPGKF activation. This is not seen in the presence of the PAR₄ mutants F347L or D349A. This experiment is a representative of three independent experiments performed in triplicates. FIG. 13D shows a scheme depicting cross-talk between GPCRs and RTK. FIGS. 13E-13G show expression of PAR₄ in breast and colon cancer tissue biopsy specimens. IHC of PAR₄. Representative sections of breast tumor tissues (FIGS. 13E and 13F) and human colon tissue sections (FIG. 13G) IHC staining, using anti PAR₄ (1:50 dilution) antibody. All images were taken using Nikon light microscope at 10× and 20× magnification. Scale bar 50 μm respectively. PAR₄ is abundantly expressed in human breast Her2/Neu+ tissue sections (FIG. 13E), as also in human breast triple-negative (TN) tumor section (FIG. 13F). PAR₄ IHC staining of human colon cancer tissue sections with metastatic invasion (FIG. 13G). In all cases, the control with no primary antibody showed very little to no staining. The experiment was carried out three times.

FIG. 14A-14C. shows the overall survival (OS) and disease metastasis free survival (DMFS) of HER2 positive breast cancer patients exhibiting high and low PAR₁/F2RL (FIG. 14A), PAR₂/F2RL1 (FIG. 14B) and PAR₄/F2RL3 (FIG. 14C). Survival analyses from TCGA data set were evaluated by Kaplan-Meier method and a log rank test was used to establish the statistical significance of the distance between curves. High and low gene expression values were defined based on the z-scores of the signals.[Prognosis-HER2+; untreated n=119; Adjusted HR: 1.9 (95% Cl: 0.9-4.3), p-0.08]

FIG. 15 shows expression of EGFR and PAR₄ in MDA-MB-231 breast cancer cell line: Western blot analysis. MDA-MB-231 cells were treated for cell lysis and separated on a SDS-PAGE. Detection of EGFR and PAR₄ in the Western blot was performed using either anti-EGFR or anti PAR₄ antibodies, respectively. This experiment is a representative of three independent experiments performed in triplicates.

FIG. 16A-16B shows the expression of EGFR in breast cancer tissue biopsy specimens. IHC of EGFR Representative sections of Her2/Neu and triple negative (TN) human breast tumor tissues. IHC staining, using anti-EGFR (1:50 dilution) antibody. Images were taken using Nikon light microscope at 20× magnification. Scale bar 50 μm respectively. EGFR in Her2/Neu (FIG. 16A) and TN tumor section (FIG. 16B). Control with no primary antibody showed very little to no expression of EGFR.

FIG. 17 shows the cross-talk between PAR₄ and EGFR. Activation of PAR₄ induces phosphorylation of EGFR. This is seen 15 min following AYPGKF the synthetic hexa peptide application for PAR₄ activation. MDA-231 cells were serum starved over night following AYPGKF PAR₄ activation (200 μM) for 5-60 min. Detection by Western blot analyses of pY-EGFR and EGFR was performed using either anti-phospho tyrosine or anti EGFR antibodies respectively. PAR₄ activation induces EGFR tyrosine phosphorylation within 15 min.

### DETAILED DESCRIPTION OF THE INVENTION

In one aspect, the present invention provides a peptide comprising an amino acid sequence SZ1Z2FRDZ3, (SEQ ID NO: 1) wherein Z1 is an amino acid selected from a hydrophobic amino acid, a modified hydrophobic amino acid, glycine, a modified glycine or histidine, Z₂ is a negatively charged amino acid and Z3 is a positively charged amino acid, wherein said peptide consists of from 7 to 25 amino acids. According to some embodiments, Z1 is an amino acid residue selected from alanine (Ala), a modified Ala, glycine (Gly), a modified Gly and histidine (His). The present invention further provides a salt and an analog of said peptide.

Thus, according to some embodiments, the present invention provides a peptide comprising an amino acid sequence SZ1Z2FRDZ3, a salt or a cyclic analog thereof, wherein said peptide or analog consists of 7 to 25 amino acids, Z1 is an amino acid residue selected from alanine (Ala), a modified Ala, glycine (Gly), a modified Gly and histidine (His); Z2 is a negatively charged amino acid; and Z3 is a positively charged amino acid. According to some embodiments, the peptide consists of 10 to 20 amino acids. According to another embodiment, the peptide consists of 10 to 15 amino acids. According to one embodiment, the peptide consists of 10, 11, 12, 13, 14 or 15 amino acids.

According to some embodiments, the present invention provides a peptide comprising an amino acid sequence SZ1Z2FRDZ3 (SEQ ID NO: 2), a salt or a cyclic analog thereof, wherein said peptide or analog consists of 7 to 25 amino acids, Z1 is an amino acid residue selected from alanine (Ala), a modified Ala, glycine (Gly), and a modified Gly; Z2 is a negatively charged amino acid; and Z3 is a positively charged amino acid. According to some embodiments, the peptide consists of 10 to 20 amino acids. According to another embodiment, the peptide consists of 10 to 15 amino acids. According to one embodiment, the peptide consists of 10, 11, 12, 13, 14 or 15 amino acids.

According to some embodiments, Z2 is an amino acid selected from aspartic acid (Asp) and glutamic acid (Glu). According to other embodiments, Z3 is an amino acid selected from lysine (Lys), arginine (Arg) and His. According to yet another embodiment, Z2 is an amino acid selected from Asp and Glu, and, Z3 is an amino acid selected from Lys, Arg and His. According to one embodiment, Z2 is Glu and Z3 is Lys. Thus, according to some embodiments, the present intention provides a peptide comprising an amino acid sequence SZ1EFRDK (SEQ ID NO: 3) wherein Z1 is an amino acid residue selected from alanine (Ala), a modified Ala, glycine (Gly), and a modified Gly, a salt or an analog thereof wherein said peptide consists of 7 to 25 amino acids. According to some embodiments, the present invention provides an analog of said peptide. According to some embodiments, Z1 is an amino acid selected from Ala, Val, Leu, Ile, Gly and His. According to some embodiments, Z1 is an amino acid selected from Ala and Gly (SEQ ID NO: 4). According to one embodiment, Z1 is Gly. According to another embodiment, Z1 is Ala.

The terms “peptide” and “polypeptide” are used herein interchangeably and refer to a chain of amino acid residues linked by peptide bonds, i.e. covalent bonds formed between the carboxyl group of one amino acid and an amino group of an adjacent amino acid. The term “peptide” refers to short sequences having up to 50 amino acids. A chain of amino acids monomers longer than 50 amino acids is referred as a “polypeptide”. Such polypeptides, when having more than 50 amino acid residues, can also be classified as proteins, more particularly, proteins of low or medium molecular weight.

According to any one of the embodiments of the present invention, the peptide is an isolated peptide. As used herein, “isolated” or “purified” when used in reference to a peptide means that the peptide has been removed from its normal physiological environment (e.g. the peptide is present as such and not in the context of the complete protein, and not in its natural compartment, namely the peptide is isolated from the cell), or is synthesized in a non-natural environment (e.g. artificially synthesized in a heterologous system).

Also included within the scope of the invention are salts of the peptides, analogs, and conjugates disclosed. “Salts” of the peptide molecules contemplated by the invention are physiologically and pharmaceutically acceptable organic and inorganic salts. Non-limitating examples of the salts of the peptides according to the present invention, include acid addition salts and base addition salts. Examples of acid addition salts include inorganic acid salts, organic acid salts, and the like. Examples of inorganic acid salts include hydrochloride, hydrobromate, sulfate, hydroiodide, nitrate, phosphate, and the like. Examples of organic acid salts include citrate, oxalate, acetate, formate, propionate, benzoate, trifluoroacetate, maleate, tartrate, methanesulfonate, benzenesulfonate, p-toluenesulfonate, and the like. Examples of base addition salts include inorganic base salts, organic base salts, and the like. Examples of inorganic base salts include sodium salt, potassium salt, calcium salt, magnesium salt, ammonium salt, and the like. Examples of organic base salts include triethyl ammonium salt, triethanol ammonium salt, pyridinium salt, diisopropylammonium salt, and the like.

According to some embodiments, the peptide consists of from 8 to 20, 9 to 18, 10 to 16 or 12 to 16 amino acids. According to one embodiment, the peptide consists of 7, 8, 9, 10, 11, 12, 13, 14, 15, 16, 17, 18, 19, 20, 21, 22, 23, 24 or 25 amino acids. According to one embodiment, the peptide consists of 7 amino acids. According to another embodiment, the peptide consists of 12 amino acids.

According to some embodiments, the peptide of the present invention comprises amino acid sequence SEQ ID NO: 2, wherein Z2 is an amino acid selected from aspartic acid (Asp) and glutamic acid (Glu). According to other embodiments, Z3 is an amino acid selected from lysine (Lys), arginine (Arg) and His. According to yet another embodiment, Z2 is an amino acid selected from aspartic acid (Asp) and glutamic acid (Glu) and Z3 is an amino acid selected from lysine (Lys), arginine (Arg) and His

According to some embodiments, the present invention provides a peptide comprising amino acid sequence X1X2SZ1EFRDKX3X4X5, wherein Z1 is an amino acid residue selected from Ala, Gly and His, X1 is a bulky hydrophobic amino acid such as Tyr, Phe, Ile and Trp, X2, X3 and X5 are each independently is a hydrophobic amino acid or Gly and X4 is a positively charged amino acid.

According to other embodiments, the present invention provides a peptide comprising amino acid sequence X1X2SZ1EFRDKX3X4X5 (SEQ ID NO: 5), wherein Z1 is an amino acid residue selected from Ala, and Gly, X1 is a bulky hydrophobic amino acid such as Tyr, Phe, Ile and Trp, X2, X3 and X5 are each independently is a hydrophobic amino acid or Gly and X4 is a positively charged amino acid. According to one embodiment, wherein Z1 is Ala. According to another embodiment, Z1 is Gly. According to one embodiment, the hydrophobic amino acid is selected from Ala, Val, Leu, Ile, Gly, Phe and Trp. According to another embodiment, the positively changed amino acid is selected from Arg, Lys and His. According to some embodiments, the peptide consists of 7 to 25 amino acids. According to another embodiment, the peptide consists of 10 to 20 amino acids. According to yet another embodiment, the peptide consists of 10 to 15 amino acids. According to one embodiment, the peptide consists of 10, 11, 12, 13, 14 or 15 amino acids.

As used herein and in any one of the embodiments of the present invention, an amino acid denoted as Z is always present, and an amino acid denoted as X may be present or absent.

According to one embodiment, the peptide comprises amino acid sequences X1X2 SZ1EFRDKX3X4X5, wherein X1 is an amino acid selected from Tyr, Phe and Trp; X2, X3 and X5 are each independently an amino acid selected from Ala, Val, Leu, Ile, and Gly; X4 is an amino acid selected from Arg and Lys, and Z1 is an amino acid selected from Ala and Gly. According to one embodiment, Z1 is Ala. According to one embodiment, Z1 is Gly. According to one embodiment, the peptide comprises the amino acid sequence YVSAEFRDKVRA (SEQ ID NO: 6). According to another embodiment, the peptide comprises amino acid sequence YVSGEFRDKVRA (SEQ ID NO: 7). According to some embodiments, the peptide consists of 7 to 25 amino acids. According to another embodiment, the peptide consists of 10 to 20 amino acids. According to yet another embodiment, the peptide consists of 10 to 15 amino acids. According to some embodiments, the peptide consists of amino acid sequence YVSAEFRDKVRA. According to other embodiments, the peptide consists of amino acid sequence YVSGEFRDKVRA.

According to any one of the above and below embodiments and aspects, the peptide is capable of inhibiting interactions of PAR protein and Pleckstrin homology (PH) domain or motif. According to some embodiments, the PAR is PAR4. Thus, according to some embodiments, the peptide of the present invention is capable of inhibiting interactions between PAR4 and PH domain. According to other embodiments, the peptide of the present invention is capable of inhibiting interactions between PAR2 and PH domain. According to some embodiments, the PH-domain is a domain of a protein comprising the PH binding domain. According to some embodiments, the protein comprising PH-binding domain are selected from Etk/Bmx, Akt/PKB, Vav, SOS1 and GAB1. According to some embodiments, the peptide of the present invention is capable of inhibiting interactions of PAR4 protein and PH binding domain of a protein selected from Etk/Bmx, Akt/PKB, Vav, SOS1 and GAB1. According to one embodiment, the peptide of the present invention is capable of inhibiting interactions of PAR4 protein and PH binding domain of Akt protein. According to some embodiments, the peptide of the present invention is capable of inhibiting interactions of PAR2 protein and PH binding domain of a protein selected from Etk/Bmx, Akt/PKB, Vav, SOS1 and GAB1. According to one embodiment, the peptide of the present invention is capable of inhibiting interactions of PAR2 protein and PH binding domain of Akt protein. The term “inhibiting interactions” has also the meaning of interfering or preventing of binding of two proteins.

According to some embodiments, the peptide is a cyclic peptide. According to other embodiments, the peptide comprises a cyclic fragment. According to a further embodiment, the peptide comprises a cyclization.

According to some embodiments, the present invention provides an analog of the peptide of the present invention. According to another embodiment, the present invention provides an analog of the peptide according to any one of the above embodiments.

The term “peptide analog”, “analog” and “sequence analog” are used herein interchangeably and refer to an analog of a peptide having at least 70% sequence identity with the original peptide, wherein the analog retains the activity of the original peptide. Thus, the terms “analog” and “active analog” may be used interchangeably. The term “analog” refers to a peptide which contains substitutions, rearrangements, deletions, additions and/or chemical modifications in the amino acid sequence of the original (parent) peptide. According to some embodiments, the peptide analog has at least 80%, at least 90% or at least 95% sequence identity to the original peptide. According to one embodiment, the analog has about 70% to about 95%, about 80% to about 90% or about 85% to about 95% sequence identity to the original peptide. According to some embodiments, the analog of the present invention comprises the sequence of the original peptide in which 1, 2, 3, 4, or 5 substitutions were made.

The substitutions of the amino acids may be a conservative or non-conservative substitution. The non-conservative substitution encompasses the substitution of one amino acid by any other amino acid. In one particular embodiment, the amino acid is substituted by a non-natural amino acid. According to another embodiment, the amino acid is substituted by a building unit (as defined hereinbelow).

The term “conservative substitution” as used herein denotes the replacement of an amino acid residue by another, without altering the overall conformation and biological activity of the peptide, including, but not limited to, replacement of an amino acid with one having similar properties (such as, for example, polarity, hydrogen bonding potential, acidic, basic, shape, hydrophobic, aromatic, and the like). Amino acids with similar properties are well known in the art. For example, according to one table known in the art, the following six groups each contain amino acids that are conservative substitutions for one another: (1) Alanine (A), Serine (S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W).

The term “amino acid” as used herein refers to an organic compound comprising both amine and carboxylic acid functional groups, which may be either a natural or non-natural amino acid. The twenty two natural amino acids are aspartic acid (Asp), tyrosine (Tyr), leucine (Leu), tryptophan (Trp), arginine (Arg), valine (Val), glutamic acid (Glu), methionine (Met), phenylalanine (Phe), serine (Ser), alanine (Ala), glutamine (Gln), glycine (Gly), proline (Pro), threonine (Thr), asparagine (Asn), lysine (Lys), histidine (His), isoleucine (Ile), cysteine (Cys), selenocysteine (Sec), and pyrrolysine (Pyl). Non-limiting examples of non-natural amino acids include diaminopropionic acid (Dap), diaminobutyric acid (Dab), ornithine (Orn), aminoadipic acid, β-alanine, 1-naphthylalanine, 3-(1-naphthyl)alanine, 3-(2-naphthyl)alanine, γ-aminobutiric acid (GABA), 3-(aminomethyl) benzoic acid, p-ethynyl-phenylalanine, p-propargly-oxy-phenylalanine, m-ethynyl-phenylalanine, p-bromophenylalanine, p-iodophenylalanine, p-azidophenylalanine, p-acetylphenylalanine, azidonorleucine, 6-ethynyl-tryptophan, 5-ethynyl-tryptophan, 3-(6-chloroindolyl)alanine, 3-(6-bromoindolyl)alanine, 3-(5-bromoindolyl)alanine, azidohomoalanine, p-chlorophenylalanine, α-aminocaprylic acid, O-methyl-L-tyrosine, N-acetylgalactosamine-α-threonine, and N-acetylgalactosamine-α-serine.

According to any one of the above embodiments, the modification of an amino acid may be a substitution by a non-natural amino acid as defined above. According to one embodiment, the non-natural amino acid is a D-amino acid. The term “D-amino acid” refers to an amino acid having the D-configuration around the α-carbon as opposite to native L-amino acid having L-conformation. As used herein, the D-amino acid in the sequence is represented by a lower case letter, whereas the L-amino acid by a capital letter.

The term “peptidomimetic” as used herein refers to a small peptide-like chain designed to mimic a peptide, which typically arises from modification of an existing peptide or by designing a similar system that mimics peptides. According to some embodiments, the term “peptide analog” and “peptidomimetic” are used interchangeably.

According to any one of the above embodiments, the present invention provides a peptide according to any one of the above embodiments in which 1, 2, 3 or 4 of amino acids is substituted by a conservative substitution. According to another embodiment, the present invention provides a peptide according to any one of the above embodiments in which 1, 2, 3 or 4 of amino acids is substituted by a non-conservative substitution, e.g. substitution with non-natural amino acids.

According to any one of the above embodiments, the analog is a cyclic analog. Thus, according to some embodiments, the present invention provides a cyclic analog of a peptide according to any one of the above embodiments.

The terms “cyclic peptide” and “cyclic analog” refers to a peptide and peptide analog, respectively, having an intramolecular bond between two non-adjacent amino acids. The cyclization can be effected through a covalent or non-covalent bond. Intramolecular bonds include, but are not limited to, backbone to backbone, side-chain to backbone and side-chain to side-chain bonds.

According to some embodiment, the present invention provides a cyclic analog comprising an amino acid sequence SZ1Z2FRDZ3 (SEQ ID NO: 38), wherein Z1 is a hydrophobic amino acid, a modified hydrophobic amino acid, glycine, a modified glycine or histidine, Z2 is a negatively charged amino acid and Z3 is a positively charged amino acid, wherein said analog consists of from 7 to 25 amino acids. According to another embodiment, the cyclic analog consists of 10 to 15 amino acids. According to one embodiment, the cyclic analog consists of 10, 11, 12, 13, 14 or 15 amino acids. According to some embodiments, the hydrophobic amino acid is selected from Ala, Val, Leu, Ile, Gly, Phe, aminobutyric acid (Abu), Norvaline (Nva) and norleucine (Nle). According to some embodiments, the positively charged amino acid is selected from arginine, lysine, diaminoacetic acid, diaminobutyric acid, diaminopropionic acid, and ornithine. According to some embodiments, the negatively charged amino acid is selected from Asp, Glu, alpha-amino adipic acid (Aad), 2-aminoheptanediacid (2-aminopimelic acid) and alpha-aminosuberic acid (Asu). According to one embodiment, Z1 is a hydrophobic amino acid selected from Ala, Val, Leu, Ile, and Phe, or Gly or His. According to another embodiment, Z2 is a negatively charged amino acid selected from Asp, Glu, and aminoadipic acid, and Z3 is a positively charged amino acid selected from Lys, Arg and His, Dap, Dab and Orn.

According to one embodiment, the cyclic analog comprises an amino acid sequence SZ1Z2FRDZ3, wherein Z1 is an amino acid selected from Ala, Val, Leu, Ile, Gly and His, Z2 is an amino acid selected from Asp and Glu, and Z3 is an amino acid selected from Lys, Arg and His, wherein said analog consists of from 7 to 25 amino acids. According to some embodiments, Z1 is Ala, Z2 is an amino acid selected from Asp and Glu, and Z3 is an amino acid selected from Lys and His. According to one embodiment, Z1 is Ala, Z2 is Asp, and Z3 is an amino acid selected from Lys and His. According to another embodiment, Z1 is Ala, Z2 is Glu, and Z3 is an amino acid selected from Lys and His. According to some embodiments, Z1 is Gly, Z2 is an amino acid selected from Asp and Glu, and Z3 is an amino acid selected from Lys and His. According to one embodiment, Z1 is Gly, Z2 is Asp, and Z3 is an amino acid selected from Lys and His. According to another embodiment, Z1 is Gly, Z2 is Glu, and Z3 is an amino acid selected from Lys and His. According to some embodiments, the cyclic analog consists of 10 to 20 amino acids. According to another embodiment, the cyclic analog consists of 10 to 15 amino acids. According to one embodiment, the cyclic analog consists of 10, 11, 12, 13, 14 or 15 amino acids.

According to one embodiment, the cyclic analog comprises amino acid sequence SAEFRDK (SEQ ID NO: 8). According to another embodiment, the cyclic analog comprises amino acid sequence SADFRDH (SEQ ID NO: 9). According to a further embodiment, the cyclic analog comprises amino acid sequence SADFRDK (SEQ ID NO: 10). According to a certain embodiment, the cyclic analog comprises amino acid sequence SHDFRDH (SEQ ID NO: 11). According to another embodiment, the cyclic analog comprises amino acid sequence SHDFRDHA (SEQ ID NO: 37).

According to some embodiments, the cyclic analog comprises an amino acid sequence X1X2SZ1Z2FRDZ3X3X4X5, wherein Z1 is an amino acid selected from Ala, Val, Leu, Ile, Gly, a modified Ala, a modified Gly, and His, Z2 is a negatively charged amino acid and Z3 is a positively charged amino acid, X2, X3 and X5, if present, are each independently an amino acid selected from Ala, Val, Leu, Ile, Gly, a modified Ala, and a modified Gly, X1, if present, is an amino acid selected from Tyr, Phe and Trp and X4 if present is an amino acid selected from Arg and Lys, wherein said cyclic analog consists of from 7 to 25 amino acids. According to some embodiments, Z1 is His. According to some embodiments, wherein Z2 is an amino acid selected from Asp and Glu, and Z3 is selected from Lys, Arg and His. According to one embodiment, Z1 is His, Z2 is an amino acid selected from Asp and Glu, and Z3 is selected from Lys and His

According to some embodiments, the cyclic analog comprises an amino acid sequence X1X2SZ1Z2FRDZ3X3X4X5 (SEQ ID NO: 12), wherein Z1 is an amino acid selected from Ala, Val, Leu, Ile, Gly, a modified Ala, and a modified Gly, Z2 is a negatively charged amino acid and Z3 is a positively charged amino acid, X2, X3 and X5, if present, are each independently an amino acid selected from Ala, Val, Leu, Ile, Gly, a modified Ala, and a modified Gly, X1, if present, is an amino acid selected from Tyr, Phe and Trp and X4 if present is an amino acid selected from Arg and Lys, wherein said cyclic analog consists of from 7 to 25 amino acids. According to some embodiments, Z1 is selected from Ala, modified Ala, Gly and a modified Gly. According to some embodiments, the cyclic analog comprises an amino acid sequence SEQ ID NO: 12, wherein Z2 is an amino acid selected from Asp and Glu, and Z3 is selected from Lys, Arg and His. According to one embodiment, Z1 is an amino acid selected from Ala and Gly, Z2 is an amino acid selected from Asp and Glu, and Z3 is selected from Lys and His (SEQ ID NO: 36).

According to another embodiment, the cyclic analog comprises the amino acid sequence SEQ ID NO: 12, wherein Z1 is selected from Ala and Gly, Z3 is selected from Lys and His and X2 if present and X3 are each Val and X1, X4 and X5 are absent.

According to one embodiment, the cyclic analog comprises the amino acid sequence SEQ ID NO: 12 wherein Z1 is Gly, Z3 is selected from Lys and His, X3 is Gly, and X1, X2, X4 and X5 are absent. According to some embodiments, the cyclic analog comprises the amino acid sequence selected from VSGEFRDKG, SGEFRDKGV, VSGEFRDKGV, YVSGEFRDKG, YVSGEFRDKGV, SGEFRDKGVR, VSGEFRDKGVR, YVSGEFRDKGVR, SGEFRDKGVRA, VSGEFRDKGVRA, and YVSGEFRDKGVRA (SEQ ID NOs: 13-23).

According to some embodiments, the present invention provides a cyclic analog comprising an amino acid sequence SZ1Z2FRDZ3X3 (SEQ ID NO: 24), wherein Z1 and X3 are each independently an amino acid residue selected from Ala, a modified Ala, Gly, and a modified Gly, Z2 is an amino acid selected from Asp and Glu, and Z3 is an amino acid selected from Lys, Arg and His. According to some embodiments, Z1 is selected from Ala or Gly. According to other embodiments, Z2 is Glu. According to yet another embodiment, Z2 is Asp. According to certain embodiments, Z3 is Lys. According to other embodiments, Z3 is His. According to one embodiment, the cyclic analog comprises amino acid sequence SGEFRDKG (SEQ ID NO: 25). According to yet another embodiment, the cyclic analog comprises amino acid sequence SGDFRDHG (SEQ ID NO: 26). According to another embodiment, the cyclic analog comprises the amino acid sequence SGDFRDKG (SEQ ID NO: 27). According to yet another embodiment, the cyclic analog comprises the amino acid sequence SGEFRDHG (SEQ ID NO: 28). According to any one of the above embodiments, a pharmaceutically acceptable salt of said cyclic analog is contemplated.

Methods for cyclization can be classified into cyclization by the formation of the amide bond between the N-terminal and the C-terminal amino acid residues, and cyclization involving the side chains of individual amino acids. The latter method includes the formation of disulfide bridges between two w-thio amino acid residues (cysteine, homocysteine), the formation of lactam bridges between glutamic/aspartic acid and lysine residues, the formation of lactone or thiolactone bridges between amino acid residues containing carboxyl, hydroxyl or mercapto functional groups, the formation of thioether or ether bridges between the amino acids containing hydroxyl or mercapto functional groups and other special methods. Lambert, et al., reviewed variety of peptide cyclization methodologies (J. Chem. Soc. Perkin Trans., 2001, 1:471-484).

Backbone cyclization is a general method by which conformational constraint is imposed on peptides. In backbone cyclization, atoms in the peptide backbone (N and/or C) are interconnected covalently to form a ring. Backbone cyclized analogs are peptide analogs cyclized via bridging groups attached to the alpha nitrogens or alpha carbonyl of amino acids. In general, the procedures utilized to construct such peptide analogs from their building units rely on the known principles of peptide synthesis; most conveniently, the procedures can be performed according to the known principles of solid phase peptide synthesis. During solid phase synthesis of a backbone cyclized peptide the protected building unit is coupled to the N-terminus of the peptide chain or to the peptide resin in a similar procedure to the coupling of other amino acids. After completion of the peptide assembly, the protective group is removed from the building unit's functional group and the cyclization is accomplished by coupling the building unit's functional group and a second functional group selected from a second building unit, a side chain of an amino acid residue of the peptide sequence, and an N-terminal amino acid residue.

As used herein the term “backbone cyclic peptide” or “backbone cyclic analog” refers to a sequence of amino acid residues wherein at least one nitrogen or carbon of the peptide backbone is joined to a moiety selected from another such nitrogen or carbon, to a side chain or to one of the termini of the peptide.

According to any of the above embodiments, the cyclization is obtained via two side chains such as to cysteines forming a Cys-Cys bond. Thus, in such embodiment, the cyclic analog comprises two Cys amino acids. According to some embodiments, each one of the Z1 and X3 are substituted with Cys. According to some embodiments, the cyclic analog comprises an amino acid sequence X1X2SZ1Z2FRDZ3X3X4X5, wherein Z1 and X3 are both Cys, Z2 is an amino acid selected from Asp and Glu, and Z3 is selected from Lys, Arg and His, X2 and X5, if present, are each independently an amino acid selected from Ala, Val, Leu, Ile, and Gly, X1, if present, is an amino acid selected from Tyr, Phe and Trp.

According to other embodiments, the cyclization is obtained via a side chain of an amino acid and a charged backbone group. According to some embodiments, the cyclic analog comprises at least one modified amino acid capable of forming a covalent bond with a backbone of the peptide analog. According to another embodiment, the cyclic analog comprises at least one modified amino acid capable of forming a covalent bond with another amino acid of the peptide to form a backbone cyclic analog.

According to a further embodiment, the cyclic analog comprises at least two modified amino acids capable of forming a covalent bond with each other to form a backbone cyclic analog. According to one embodiment, the cyclic analog comprises at least two non-contiguous modified amino acids capable of forming a covalent bond with each other to form a backbone cyclic analog.

According to any one of the above embodiments, the covalent bond is selected from ester, amid, urea, thiourea, disulfide and guanoidino bond. As used herein the terms “urea bond” refers to —NH—CO—NH-bond. The terms “urea bond”, “thiourea bond”, and “guanoidino bond” refer to bonding that are resulted in urea, thiourea and guanoidino groups, respectively.

According to some embodiments, the cyclic analog comprises two Na-w-functionalized amino acid derivatives, namely two building units, connected to form a backbone cyclic analog. According to some embodiments, the two Na-w-functionalized amino acid derivatives are non-contiguous amino acids. According to some embodiments, any Na-w-functionalized amino acid derivative may be used according to the teaching of the present invention.

The term “building unit” (BU) refers to a Na-w-functionalized or an Ca-w-functionalized derivative of amino acids. Use of such building units permits different length and type of linkers and different types of moieties to be attached to the scaffold. This enables flexible design and easiness of production using conventional and modified solid-phase peptide synthesis methods known in the art.

According to some embodiments, the BU is an N^{α}-ω-functionalized derivative of amino acids having the following formula:

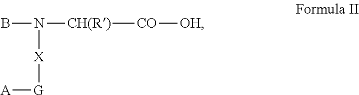

wherein X is a spacer group selected from the group consisting of alkylene, substituted alkylene, arylene, cycloalkylene and substituted cycloalkylene; R′ is an amino acid side chain, optionally bound with a specific protecting group, or absent; B is a protecting group selected from the group consisting of alkyloxy, substituted alkyloxy, or aryl carbonyls; and G is a functional group selected from the group consisting of amines, thiols, alcohols, carboxylic acids and esters, aldehydes, alcohols and alkyl halides; and A is a specific protecting group of G.

According to some embodiments, building units are the N^{α}-ω-functionalized amino acid derivatives wherein X is alkyl; G is a thiol group, an amino group or a carboxyl group; and R′ is the side chain of an amino acid. Further preferred are co-functionalized amino acid derivatives wherein R′ is protected with a specific protecting group.

According to more specific embodiments, the building units are N^{α}-ω-functionalized amino acid derivatives wherein G is an amino group, a carboxyl group, or a thiol group of the following formulae:

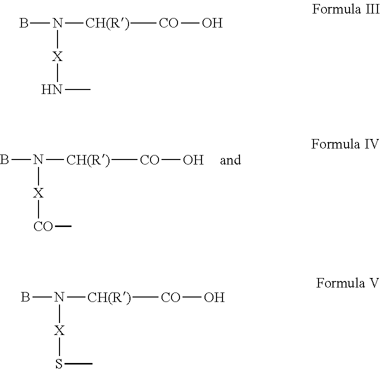

The terms “alkyl” and “alkylenyl” are used herein interchangeably and refer to both branched and straight-chain saturated aliphatic hydrocarbon groups having one to 20 carbon atoms.

The term “alkenyl” as used herein refers to hydrocarbon chains of either a straight or branched configuration having two to 20 carbon atoms and one or more unsaturated carbon-carbon bonds which may occur in any stable point along the chain, such as ethenyl, propenyl, and the like.

The term “alkynyl” as used herein refers to hydrocarbon chains of either a straight or branched configuration having from two to 20 carbon atoms and one or more triple carbon-carbon bonds which may occur in any stable point along the chain, such as ethynyl, propynyl, and the like.

As used herein and in the claims, the term “aryl” is intended to mean any stable 5- to 7-membered monocyclic or bicyclic or 7- to 14-membered bicyclic or tricyclic carbon ring, any of which may be saturated, partially unsaturated or aromatic, for example, phenyl, naphthyl, indanyl, or tetrahydronaphthyl etc.

As used herein and in the claims, “alkyl halide” is intended to include both branched and straight-chain saturated aliphatic hydrocarbon groups having the one to ten carbon atoms, wherein 1 to 3 hydrogen atoms have been replaced by a halogen atom such as Cl, F, Br, and I.

The terms “cycloalkyl” and “cycloalkenyl” are used herein interchangeably and refers to cyclic saturated aliphatic radicals containing 3 to 12 carbon atoms in the ring, such as cyclopropyl, cyclobutyl, cyclopentyl, cyclohexyl, or cyclododecyl. Such cycloalkyl ring systems may be fused to other cycloalkls, such in the case of cis/trans decalin.

According to one embodiment, the alkyl is a straight alkyl having the formula (CH2)n wherein n is an integer between 1 to 20 and R′ is a residue of an amino acid selected from Gly, Val, and Ala. As such the building unit comprising R′ of Gly is referred as Gly-BU, the BU comprising the R′ of a Val is referred as Val-BU, and building unit comprising R′ of Ala is referred as Ala-BU. The alkyl group of the building unit permits different length of linkers. According to some embodiments, n is an integer between 2 to 10, 3 to 9, 4 to 8 or 5 to 6. Thus the BU comprises a (C1-C10)alkyl, (C2-C8)alkyl, (C1-C10)alkyl, or (C3-C6)alkyl. According to another embodiment, the BU comprises C3-alkyl, C4-alkyl, C5-alkyl or C6-alkyl. According to some embodiments, the backbone cyclic analog comprises at least two modified amino acids selected from Ala-BU, Gly-BU and Val-BU.

In general, the procedures utilized to construct backbone cyclic molecules and their building units rely on the known principles of peptide synthesis and peptidomimetic synthesis; most conveniently, the procedures can be performed according to the known principles of solid phase peptide synthesis. Some of the methods used for producing Nα-ω) building units and for their incorporation into peptidic chain are disclosed in U.S. Pat. Nos. 5,811,392; 5,874,529; 5,883,293; 6,051,554; 6,117,974; 6,265,375, 6,355613, 6,407059, 6,512092 and international applications WO 95/33765; WO 97/09344; WO 98/04583; WO 99/31121; WO 99/65508; WO 00/02898; WO 00/65467 WO 02/062819 and WO 2017/212477.

The backbone cyclic peptides of the present invention may be produced by any method known in the art enabling the creation of such molecules. Synthetic methods include exclusive solid phase synthesis, partial solid phase synthesis, fragment condensation, or classical solution synthesis. Solid phase peptide synthesis procedures are well known to one skilled in the art and. In some embodiments, synthetic peptides are purified by preparative high performance liquid chromatography and the peptide sequence is confirmed via amino acid sequencing by methods known to one skilled in the art.

According to some embodiments, the BUs in the peptide form a covalent bond. According to some embodiments, the binding of two BUs forms a group selected an ester, amid, urea, thiourea, disulfide and guanoidino group. Thus, such cyclic analog comprises a group selected from ester, amid, urea, thiourea, disulfide and guanoidino group between two alkyls of the BUs. According to some embodiments, the peptide comprises Gly-BUs cyclized via urea bond to form a backbone cyclic peptide analog.

According to other embodiments, the cyclic analog comprises two Ca-functionalized amino acid derivatives. According to some embodiments, the cyclic analog comprises at least two non-contiguous modified amino acids capable of forming a covalent bond with each other to form a backbone cyclic analog. According to some embodiments, the two modified amino acids are N^{α}-ω-functionalized amino acid derivatives capable of forming a covalent bond with another amino acid residue or with the terminus of the peptide (building unit, BU). According to yet another embodiment, the covalent bond is selected from an ester, amid, urea, thiourea, disulfide and guanoidino bond.

According to some embodiment, the present invention provides a cyclic analog comprising an amino acid sequence SEQ ID NO: 12, wherein, wherein Z1 and X3 are each independently a modified amino acid, Z2 is a negatively charged amino acid, Z3 is a positively charged amino acid and X1, X2, X4 and X5 are absent. According to some embodiments, the modified amino acids is selected from N^{α}-ω-functionalized and Ca-co-functionalized amino acid derivative. According to some embodiments, the modified amino acids are N^{α}-ω-functionalized amino acid derivatives (SEQ ID NO: 34). According to some embodiments, Z1 and X3 are each independently an amino acid selected from a modified Ala and a modified Gly. According to some embodiment, the present invention provides a cyclic analog comprising the sequence SZ1Z2FRDZ3X3 (SEQ ID NO: 35), wherein Z1 and X3 are each independently an amino acid selected from a modified Ala and a modified Gly, Z2 is selected from Asp and Glu and Z3 is selected from Lys and His. According to some embodiments, the modified amino acids are Na-co-functionalized amino acid derivatives. According to some embodiments, the Z1 and X3 are each independently selected from a Gly-BU and Ala-BU. According to another embodiment, the modified amino acids form a covalent bond is selected from an ester, amid, urea, thiourea, disulfide and guanoidino bond. Therefore, according to some embodiments, the cyclic analog is a backbone cyclic analog.

According to some embodiment, the cyclic analog comprises amino acid sequence SZ1Z2FRDZ3X3 (SEQ ID NO: 29), wherein Z1 and X3 are each independently selected from a Gly-BU and Ala-BU, Z2 is selected from Asp and Glu and, Z3 is selected from Lys and His. According to other embodiments, Z1 and X3 are each Ala-building unit. According to one embodiments, Z1 and X3 are each Gly-building unit. According to some embodiments, the Z1 and the X3 are covalently bound via an urea group. According to other embodiments, the Z1 and X3 are building units each individually comprising a (C1-C10) alkyl. According to another embodiment, the Z1 comprises 3, 4, 5 or 6 (CH)2 groups. According to another embodiment, X3 comprises 3, 4, 5 or 6 (CH)2 groups. According to some embodiments, the cyclic analog is a backbone cyclic analog.

According to some embodiment, the cyclic analog comprises Z1 and X3, wherein the Z1 and X3 each individually a building unit comprising a (C3-C6) alkyl. According to further embodiment, the cyclic analog comprises Z1 and X3, wherein the Z1 and X3 each individually a building unit comprising a (C3-05) alkyl. According to some embodiment, the cyclic analog comprises Z1 and X3, wherein the Z1 and X3 each individually a building unit comprising a (C3-C6) alkyl. According to one embodiment, Z1 comprises C3 alkyl and X3 comprises an alkyl selected from C3, C4, C5 and C6 alkyl. According to another embodiment, Z1 comprises C4 alkyl and X3 comprises an alkyl selected from C3, C4, C5 and C6 alkyl. According to yet another embodiment, Z1 comprises C5 alkyl and X3 comprises an alkyl selected from C3, C4, C5 and C6 alkyl. According to a further another embodiment, Z1 comprises C6 alkyl and X3 comprises an alkyl selected from C3, C4, C5 and C6 alkyl. According to some embodiments, the building unit are bound via a covalent bond to form backbone cyclization.

According to the teaching of the present invention the term “comprises an alkyl” as used with respect to a building unit means refers to an alkyl at position X as presented in Formulas II-V.

According to some embodiments, the backbone cyclic analog comprises an amino acid sequence selected from SZ1EFRDKX3 (SEQ ID NO: 30) SZ1DFRDHX3 (SEQ ID NO: 31), SZ1EFRDHX3 (SEQ ID NO: 32), and SZ1DFRDKX3 (SEQ ID NO: 33), wherein Z1 and X3 are both Gly building units each comprising a (C2-C6) alkyl and are covalently bound via urea group. According to some embodiments, the Z1 and X3 are both Gly building units each comprising a (C3-05) alky covalently bound via urea group. According to other embodiments, the Z1 and X3 are both Gly building units each comprising a (C3-C6) alky covalently bound via urea group. The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods. According to some embodiments, the backbone cyclic analog consists of an amino acid sequence selected from SEQ ID NO: 30-33, wherein Z1 and X3 are both Gly building units each comprising a (C2-C6) alkyl and are covalently bound via urea group.

According to one embodiment, the backbone cyclic analog comprises amino acid sequence SZ1EFRDKX3 (SEQ ID NO: 30), wherein Z1 and X3 are both Gly building unit each comprising a (C3-C6) alky covalently bound via urea group. According to one embodiment, Z1 comprises C3 alkyl and X3 comprises an alkyl selected from C3, C4, C5 and C6 alkyl. According to another embodiment, Z1 comprises C4 alkyl and X3 comprises an alkyl selected from C3, C4, C5 and C6 alkyl. According to yet another embodiment, Z1 comprises C5 alkyl and X3 comprises an alkyl selected from C3, C4, C5 and C6 alkyl. According to a further another embodiment, Z1 comprises C6 alkyl and X3 comprises an alkyl selected from C3, C4, C5 and C6 alkyl. According to one embodiment, Z1 comprises C3 alkyl and X3 comprises C3 alkyl. According to one embodiment, Z1 comprises C3 alkyl and X3 comprises C4 alkyl. According to one embodiment, Z1 comprises C3 alkyl and X3 comprises C5 alkyl. According to one embodiment, Z1 comprises C4 alkyl and X3 comprises C3 alkyl. According to one embodiment, Z1 comprises C4 alkyl and X3 comprises C4 alkyl. According to one embodiment, Z1 comprises C4 alkyl and X3 comprises C5 alkyl. According to one embodiment, Z1 comprises C5 alkyl and X3 comprises C5 alkyl. According to some embodiments, the backbone cyclic analog has the structure of Formula I

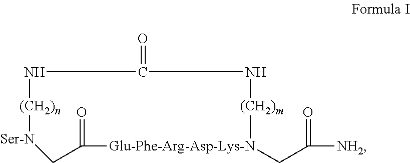

wherein n and m are each independently an integer between 3 and 6. According to some embodiments, n is 2 and m is selected from 3, 4, 5 and 6. According to other embodiments, n is 3 and m is selected from 2, 3, 4, 5 and 6. According to other embodiments, n is 4 and m is selected from 2, 3, 4, 5 and 6. According to further embodiments, n is 5 and m is selected from 2, 3, 4, 5 and 6. According to yet another embodiment, n is 6 and m is selected from 2, 3, 4 and 5. According to one embodiment, the peptidomimetic has the structure of Formula I, wherein m=n=4. In some embodiments of the invention, the peptidomimetic having the structure of Formula I, wherein m=n=4 is referred also as PAR(4-4) and PAR 4×4 analog or inhibitor.

According to one embodiment, the backbone cyclic analog comprises amino acid sequence SZ1DFRDHX3 (SEQ ID NO: 31), wherein Z1 and X3 are both Gly building unit each comprising a (C3-C6) alky covalently bound via urea unit. According to one embodiment, Z1 comprises C3 alkyl and X3 comprises an alkyl selected from C3, C4, C5 and C6 alkyl. According to another embodiment, Z1 comprises C4 alkyl and X3 comprises an alkyl selected from C3, C4, C5 and C6 alkyl. According to yet another embodiment, Z1 comprises C5 alkyl and X3 comprises an alkyl selected from C3, C4, C5 and C6 alkyl. According to a further another embodiment, Z1 comprises C6 alkyl and X3 comprises an alkyl selected from C3, C4, C5 and C6 alkyl. According to one embodiment, Z1 comprises C3 alkyl and X3 comprises C3 alkyl. According to one embodiment, Z1 comprises C3 alkyl and X3 comprises C4 alkyl. According to one embodiment, Z1 comprises C3 alkyl and X3 comprises C5 alkyl. According to another embodiment, Z1 comprises C4 alkyl and X3 comprises C3 alkyl. According to yet another embodiment, Z1 comprises C4 alkyl and X3 comprises C4 alkyl. According to a further embodiment, Z1 comprises C4 alkyl and X3 comprises C5 alkyl. According to one embodiment, Z1 comprises C5 alkyl and X3 comprises C5 alkyl.

According to one embodiment, the backbone cyclic analog comprises an amino acid sequence selected from SZ1EFRDHX3 (SEQ ID NO: 32) and SZ1DFRDKX3 (SEQ ID NO: 33), wherein Z1 and X3 are both Gly building unit each comprising a (C3-C6) alky covalently bound via urea unit. According to one embodiment, Z1 comprises C3 alkyl and X3 comprises an alkyl selected from C3, C4, C5 and C6 alkyl. According to another embodiment, Z1 comprises C4 alkyl and X3 comprises an alkyl selected from C3, C4, C5 and C6 alkyl. According to yet another embodiment, Z1 comprises C5 alkyl and X3 comprises an alkyl selected from C3, C4, C5 and C6 alkyl. According to another embodiment, Z1 comprises C6 alkyl and X3 comprises an alkyl selected from C3, C4, C5 and C6 alkyl. According to one embodiment, Z1 comprises C3 alkyl and X3 comprises C3 alkyl. According to one embodiment, Z1 comprises C3 alkyl and X3 comprises C4 alkyl. According to one embodiment, Z1 comprises C3 alkyl and X3 comprises C5 alkyl. According to one embodiment, Z1 comprises C4 alkyl and X3 comprises C3 alkyl. According to one embodiment, Z1 comprises C4 alkyl and X3 comprises C4 alkyl. According to one embodiment, Z1 comprises C4 alkyl and X3 comprises C5 alkyl. According to one embodiment, Z1 comprises C5 alkyl and X3 comprises C5 alkyl.

According to some embodiment, the ring of the cyclic analog comprises from 20 to 50 atoms. According to other embodiments, the ring of the cyclic analog comprises from 22 to 48, from 25 to 45, from 28 to 43, from 30 to 40, from 32 to 38, or from 34 to 36 atoms. According to some embodiments, the ring of the cyclic analog comprises from 27 to 33 atoms, from 28 to 32 or from 39 to 31 atoms. According to some embodiments, the ring of the cyclic analog comprises 30 atoms. According to some embodiments, the ring of the cyclic analog comprises 29 atoms. According to some embodiments, the ring of the cyclic analog comprises 31 atoms. According to some embodiments, the ring of the cyclic analog comprises 28 atoms. According to some embodiments, the ring of the cyclic analog comprises 32 atoms. The term comprises has the meaning of consists of and may be replaced by it. Thus, according to some embodiments, the ring of the cyclic analog consists of from 20 to 50, from 22 to 48, from 25 to 45, from 28 to 43, from 30 to 40, from 32 to 38 or from 34 to 36 atoms, 28, 29, 30, 31 or 32 atoms.

According to some embodiments, the pharmaceutically acceptable salt of said cyclic analog is contemplated.

According to any one of the above embodiments, the present invention provides a conjugate of the peptide, peptide analog, cyclic peptide or cyclic analog of the present invention. According to one embodiment, the present invention provides a conjugate of the peptide of the present invention. According to another embodiment, the present invention provides a conjugate of the analog of the present invention. According to a certain embodiment, the present invention provides a conjugate of the cyclic analog of the present invention. According to some embodiments, the conjugate is PEG conjugate. According to other embodiment, the peptide, peptide analog or cyclic peptide analog is conjugated with a permeability enhancing moiety. According to one embodiment, the present invention provides a conjugate of the cyclic analog comprising an amino acid sequence selected from SEQ ID NO: 29-33. According to one embodiment, the present invention provides a conjugate of the cyclic analog consisting of an amino acid sequence selected from SEQ ID NO: 29-33. According to one embodiment, the present invention provides a conjugate of the cyclic analog having the structure of Formula I.

The term “permeability-enhancing moiety” refers to any moiety known in the art to facilitate actively or passively or enhance the permeability of the compound through body barriers or into the cells. Non-limitative examples of permeability-enhancing moiety include hydrophobic moieties such as fatty acids, steroids and bulky aromatic or aliphatic compounds; moieties which may have cell-membrane receptors or carriers, such as steroids, vitamins and sugars, natural and non-natural amino acids and transporter peptides, nanoparticles and liposomes. The term “permeability” refers to the ability of an agent or substance to penetrate, pervade, or diffuse through a barrier, membrane, or a skin layer.

According to any one of the above and below embodiments and aspects, the cyclic analog of the present invention is capable of inhibiting interactions of PAR protein and Pleckstrin homology (PH) domain or motif. According to some embodiments, the PAR is PAR4. Thus, according to some embodiments, the cyclic analog of the present invention is capable of inhibiting interactions between PAR4 and PH domain. According to other embodiments, the cyclic analog of the present invention is capable of inhibiting interactions between PAR2 and PH domain. According to some embodiments, the PH-domain is a domain of a protein comprising the PH binding domain. According to some embodiments, the protein comprising PH-binding domain are selected from Etk/Bmx, Akt/PKB, Vav, SOS1 and GAB1. According to some embodiments, the cyclic analog of the present invention is capable of inhibiting interactions of PAR4 protein and PH binding domain of a protein selected from Etk/Bmx, Akt/PKB, Vav, SOS1 and GAB1. According to one embodiment, the cyclic analog of the present invention is capable of inhibiting interactions of PAR4 protein and PH binding domain of Akt protein. According to some embodiments, the cyclic analog of the present invention is capable of inhibiting interactions of PAR2 protein and PH binding domain of a protein selected from Etk/Bmx, Akt/PKB, Vav, SOS1 and GAB1. According to one embodiment, the cyclic analog of the present invention is capable of inhibiting interactions of PAR2 protein and PH binding domain of Akt protein.

According to another aspect, the present invention provides a pharmaceutical composition comprising the peptide, cyclic peptide, analog or cyclic analog of the present invention or a salt thereof, and a pharmaceutically acceptable excipient. According to another embodiment, the pharmaceutical composition comprises a conjugate of the peptide, cyclic peptide, analog or cyclic analog of the present invention.

The term “pharmaceutical composition” as used herein refers to a composition comprising at least one active agent as disclosed herein optionally formulated together with one or more pharmaceutically acceptable carriers.

According to one embodiment, the present invention provides a pharmaceutical composition comprising the peptide of the present invention. According to other embodiments, the pharmaceutical composition comprises a peptide comprising an amino acid sequence SZ1Z2FRDZ3 (SEQ ID NO: 2), a salt or a cyclic analog thereof, wherein said peptide consists of 7 to 25 amino acids, Z1 is an amino acid residue selected from alanine (Ala), a modified Ala, glycine (Gly), and a modified Gly; Z2 is a negatively charged amino acid; and Z3 is a positively charged amino acid. According to one embodiment, the peptide comprises an amino acid sequence SZ1EFRDK, wherein Z1 is an amino acid selected from Ala, Val, Leu, Ile, Gly and His said peptide consists of 7 to 25 amino acids. According to one embodiment, Z1 is Gly. According to another embodiment, Z1 is Ala. According to one embodiment, the peptide comprises or consists of amino acid sequence YVSAEFRDKVRA. According to another embodiment, the peptide comprises or consists of amino acid sequence YVSGEFRDKVRA.

According to one embodiment, the present invention provides a pharmaceutical composition comprising a cyclic analog of the peptide of the present invention. According to one embodiment, a peptide analog comprising amino acid sequence SZ1Z2FRDZ3, wherein Z1 is a hydrophobic amino acid, a modified hydrophobic amino acid, glycine, or a modified glycine, Z2 is a negatively charged amino acid and Z3 is a positively charged amino acid, wherein said analog consists of from 7 to 25 amino acids. According to one embodiment, the analog of the peptide comprises amino acid sequence SZ1Z2FRDZ3, wherein Z1 is an amino acid selected from Ala, Val, Leu, Ile, Gly and His, Z2 is an amino acid selected from Asp and Glu, and Z3 is an amino acid selected from Lys, Arg and His, wherein said analog consists of from 7 to 25 amino acids. According to some embodiments, the analog is a cyclic analog. Thus, according to some embodiments, the present invention provides a pharmaceutical composition comprising a cyclic analog comprising amino acid sequence SZ1Z2FRDZ3, wherein Z1 is an amino acid selected from Ala, Val, Leu, Ile, Gly and His, Z2 is an amino acid selected from Asp and Glu, and Z3 is an amino acid selected from Lys, Arg and His, wherein said analog consists of from 7 to 25 amino acids. According to some embodiments, Z1 is an amino acid selected from Ala, Gly, Val, Leu, and Ile, According to some embodiments, the pharmaceutical composition comprises a cyclic analog comprising an amino acid sequence selected from SAEFRDK, SADFRDH, SADFRDK and SHDFRDH. According to another embodiment, the pharmaceutical composition comprises a cyclic analog comprising an amino acid sequence selected from SAEFRDK, SADFRDH, and SADFRDK.

According to other embodiments, the pharmaceutical composition comprises a cyclic analog comprises an amino acid sequence X1X2SZ1Z2FRDZ3X3X4X5, wherein Z1 is an amino acid selected from Ala, Val, Leu, Ile, Gly, a modified Ala, and a modified Gly, Z2 is an amino acid selected from Asp and Glu, and Z3 is selected from Lys, Arg and His, X2, X3 and X5, if present, are each independently an amino acid selected from Ala, Val, Leu, Ile, Gly, a modified Ala, and a modified Gly, X1, if present, is an amino acid selected from Tyr, Phe and Trp. According to certain embodiments, the pharmaceutical composition comprises a cyclic analog comprises an amino acid sequence X1X2SZ1Z2FRDZ3X3X4X5, wherein Z1 is an amino acid selected from Ala, Gly, a modified Ala, and a modified Gly, Z2 is an amino acid selected from Asp and Glu, and Z3 is selected from Lys, Arg and His, X2, X3 and X5, if present, are each independently an amino acid selected from Ala, Val, Leu, Ile, Gly, a modified Ala, and a modified Gly, X1, if present, is an amino acid selected from Tyr, Phe and Trp. According to some embodiments, the cyclic analog comprises amino acid sequence selected from

SGEFRDKG, SGDFRDHG, VSGEFRDKG, SGEFRDKGV,
VSGEFRDKGV, YVSGEFRDKG, YVSGEFRDKGV, SGEFRDKGVR,
VSGEFRDKGVR,YVSGEFRDKGVR, SGEFRDKGVRA,
VSGEFRDKGVRA,
and
YVSGEFRDKGVRA.

According to yet another embodiment, the present invention provides a pharmaceutical composition comprising a cyclic analog comprising an amino acid sequence SZ1Z2FRDZ3X3 (SEQ ID NO: 34), wherein Z1 and X3 are each independently an Na-w-functionalized amino acid derivative building unit, Z2 is a negatively charged amino acid and Z3 is a positively charged amino acid. According to yet another embodiment, the present invention provides a pharmaceutical composition comprising a cyclic analog comprising amino acid sequence SZ1Z2FRDZ3X3, wherein Z1 and X3 are each independently selected from a Gly and Ala, building unit, Z2 is selected from Asp and Glu, and Z3 is selected from Lys and His. According to other embodiments, Z1 and X3 are each Ala-building unit. According to one embodiments, Z1 and X3 are each Gly-building unit. According to some embodiments, the Z1 and the X3 are covalently bound via an urea group. According to one embodiments, the Z1 and X3 are each individually comprise a (C1-C10) alkyl. According to some embodiments, the backbone cyclic analog comprises amino acid sequence selected from SZ1EFRDKX3 SZ1DFRDHX3, SZ1EFRDHX3, and SZ1DFRDKX3, wherein Z1 and X3 are both Gly building unit each comprising a (C3-C6) alky covalently bound via urea group. According to one embodiment, Z1 comprises C3 alkyl and X3 comprises an alkyl selected from C3, C4, C5 and C6 alkyl; or Z1 comprises C4 alkyl and X3 comprises an alkyl selected from C3, C4, C5 and C6 alkyl; or Z1 comprises C5 alkyl and X3 comprises an alkyl selected from C3, C4, C5 and C6 alkyl; or Z1 comprises C6 alkyl and X3 comprises an alkyl selected from C3, C4, C5 and C6 alkyl. According to one embodiment, Z1 and X3 are comprise C4 alky. According to one embodiment, the present invention provides a pharmaceutical composition comprising a backbone cyclic analog having structure of Formula I. According to any one of the above embodiment, the pharmaceutical composition comprises a pharmaceutically acceptable salt of said peptide or analog. According to some embodiments, the pharmaceutical composition comprises a conjugate of said peptides or said peptide analogs e.g. cyclic analogs. According to one embodiment, the conjugate is a conjugate of the cyclic analog comprising an amino acid sequence selected from SEQ ID NO: 29-33. According to one embodiment, the present invention provides a pharmaceutical composition comprising a conjugate of the cyclic analog having the structure of Formula I.

According to any one of the above embodiments, the term “comprises” encompasses the term “consisting of” and may be replaced by it.

Formulation of the pharmaceutical composition may be adjusted according to its applications. In particular, the pharmaceutical composition may be formulated using a method known in the art so as to provide a rapid, continuous or delayed release of the active ingredient after administration to mammals. For example, the formulation may be any one selected from among plasters, granules, lotions, liniments, lemonades, aromatic waters, powders, syrups, ophthalmic ointments, liquids and solutions, aerosols, extracts, elixirs, ointments, fluidextracts, emulsions, suspensions, decoctions, infusions, ophthalmic solutions, tablets, suppositories, injections, spirits, capsules, creams, troches, tinctures, pastes, pills, and soft or hard gelatin capsules.

The pharmaceutical composition of the present invention may be administered by any known method.

The terms “administering” or “administration of” refer to any know method of administration and include administration intravenously, arterially, intradermally, intramuscularly, intraperitonealy, intravenously, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. Thus, the pharmaceutical composition of the present invention is formulated to be administered by any one of the above routes of administration.

The composition for oral administration may be in a form of tablets, troches, lozenges, aqueous or oily suspensions, solutions, dispersible powders or granules, emulsions, hard or soft capsules, or syrups or elixirs. Pharmaceutical compositions intended for oral use may be prepared according to any method known to the art for the manufacture of pharmaceutical compositions and may further comprise one or more agents selected from sweetening agents, flavoring agents, coloring agents and preserving agents in order to provide pharmaceutically elegant and palatable preparations. Tablets contain the active agent in admixture with non-toxic pharmaceutically acceptable excipients, which are suitable for the manufacture of tablets. These excipients may be, e.g., inert diluents such as calcium carbonate, sodium carbonate, lactose, calcium phosphate, or sodium phosphate; granulating and disintegrating agents, e.g., corn starch or alginic acid; binders; and lubricating agents. The tablets are preferably coated utilizing known techniques to delay disintegration and absorption in the gastrointestinal tract and thereby provide an extended release of the drug over a longer period.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The compositions may contain other active compounds providing supplemental, additional, or enhanced therapeutic functions. solid carriers or excipients such as, for example, lactose, starch or talcum or liquid carriers such as, for example, water, fatty oils or liquid paraffin.

Other carriers or excipients which may be used include, but are not limited to, materials derived from animal or vegetable proteins, such as the gelatins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; polypeptide/protein or polysaccharide complexes such as gelatin-acacia complexes; sugars such as mannitol, dextrose, galactose and trehalose; cyclic sugars such as cyclodextrin; inorganic salts such as sodium phosphate, sodium chloride and aluminium silicates; and amino acids having from 2 to 12 carbon atoms and derivatives thereof such as, but not limited to, glycine, L-alanine, L-aspartic acid, L-glutamic acid, L-hydroxyproline, L-isoleucine, L-leucine and L-phenylalanine. Each possibility represents a separate embodiment of the present invention.

Solutions or suspensions used for parenteral, intradermal, or subcutaneous application typically include the following components: a sterile diluent such as water for injection, saline solution, fixed oils, polyethylene glycols, glycerin, propylene glycol (or other synthetic solvents), antibacterial agents (e.g., benzyl alcohol, methyl parabens), antioxidants (e.g., ascorbic acid, sodium bisulfite), chelating agents (e.g., ethylenediaminetetraacetic acid), buffers (e.g., acetates, citrates, phosphates), and agents that adjust tonicity (e.g., sodium chloride, dextrose). The pH can be adjusted with acids or bases, such as hydrochloric acid or sodium hydroxide, for example. The parenteral preparation can be enclosed in ampules, disposable syringes or multiple dose glass or plastic vials. The term “parenteral” refers to subcutaneous, intracutaneous, intravenous, intramuscular, intraarticular, intraarterial, intrasynovial, intrasternal, intrathecal, intralesional, intraperitoneal and intracranial injection, as well as various infusion techniques.

Pharmaceutical compositions adapted for parenteral administration include, but are not limited to, aqueous and non-aqueous sterile injectable solutions or suspensions, which can contain antioxidants, buffers, bacteriostats and solutes that render the compositions substantially isotonic with the blood of an intended recipient. Such compositions can also comprise water, alcohols, polyols, glycerin and vegetable oils, for example. Extemporaneous injection solutions and suspensions can be prepared from sterile powders, granules and tablets. Such compositions preferably comprise a therapeutically effective amount of a compound of the invention and/or other therapeutic agent(s), together with a suitable amount of carrier so as to provide the form for proper administration to the subject.

According to some embodiments, the pharmaceutical composition of the present invention is for use in treating a disease mediated by a protease-activated receptor (PAR).

The terms “protease-activated receptor” and “PAR” are used herein interchangeably and refer to the protein subfamily of related G protein-coupled receptors that are activated by cleavage of their N-terminal extracellular domain. The subfamily comprises 4 known protease-activated receptors: PAR1, PAR2, PAR3, and PAR4. The terms “PAR4” and “PAR4” are used herein interchangeably.

According to one embodiment, the pharmaceutical of the present invention is for use in treating a disease mediated by PAR1. According to another embodiment, the pharmaceutical of the present invention is for use in treating a disease mediated by PAR2. According to yet another embodiment, the pharmaceutical of the present invention is for use in treating a disease mediated by PAR3. According to some embodiments, the pharmaceutical of the present invention is for use in treating a disease mediated by PAR4. According to some embodiments, the pharmaceutical of the present invention is for use in treating a disease mediated by PAR4 or PAR2.

The term “mediated by a PAR” as used herein means that a process, physiological condition, disease, disorder or condition is modulated by, caused by and/or has some biological basis, that directly or indirectly involves or includes PAR protein activity such as signal transduction. Thus, modulating the activity PAR such as inhibiting its interaction with other proteins e.g. by peptides or analogs according to the present invention has a beneficial effect on a disease or a condition.

According to some embodiments, the disease mediated by PAR, e.g. by PAR4, is cancer. According to one embodiment, the pharmaceutical of the present invention is for use in treating cancer. According to another embodiment, treating cancer comprises killing cancer stem cells. According to some embodiments, the disease mediated via PAR2, is cancer.

Therefore, according to some embodiments, the pharmaceutical composition of the present intention is for use in treating cancer.

The term “cancer” comprises cancerous diseases or a tumor being treated or prevented that is selected from the group comprising, but not limited to, carcinomas, melanoma, sarcoma, mammary carcinomas, melanoma, skin neoplasms, lymphoma, leukemia, gastrointestinal tumors, including colon carcinomas, stomach carcinomas, pancreas carcinomas, colon cancer, small intestine cancer, ovarian carcinomas, cervical carcinomas, lung cancer, prostate cancer, kidney cell carcinomas and/or liver metastases. According to certain embodiments, the cancer is a carcinoma. According to some embodiments, the cancer is colon cancer. According to other embodiments, the cancer is breast cancer.

The term “cancer stem cells (CSCs)” as used herein refers to cancer cells (found within tumors or hematological cancers) that possess characteristics associated with normal stem cells, specifically the ability to give rise to all cell types found in a particular cancer sample, having pluripotency and self-renewal ability. CSCs may generate tumors through the stem cell processes of self-renewal and differentiation into multiple cell types. The terms “tumor stem-like cells” are “tumor initiating cells” are essentially synonymous to the term “cancer stem cells” and may be used interchangeably.

According to one embodiment, the pharmaceutical composition of the present invention is for use in treating carcinoma. The term “carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum. According to another embodiment, the pharmaceutical composition of the present invention is for use in treating breast cancer. According to yet another embodiment, the pharmaceutical composition of the present invention is for use in treating colon cancer.

The term “treating” a condition or patient refers to taking steps to obtain beneficial or desired results, including clinical results. Beneficial or desired clinical results include, but are not limited to, or ameliorating abrogating, substantially inhibiting, slowing or reversing the progression of a disease, condition or disorder, substantially ameliorating or alleviating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and/or (e) limiting recurrence of symptoms in patients that were previously asymptomatic for the disorder(s).

The term “treating cancer” as used herein should be understood to e.g. encompass treatment resulting in a decrease in tumor size; a decrease in rate of tumor growth; stasis of tumor size; a decrease in the number of metastasis; a decrease in the number of additional metastasis; a decrease in invasiveness of the cancer; a decrease in the rate of progression of the tumor from one stage to the next; inhibition of tumor growth in a tissue of a mammal having a malignant cancer; control of establishment of metastases; inhibition of tumor metastases formation; regression of established tumors as well as decrease in the angiogenesis induced by the cancer, inhibition of growth and proliferation of cancer cells and so forth. The term “treating cancer” as used herein should also be understood to encompass prophylaxis such as prevention as cancer reoccurs after previous treatment (including surgical removal) and prevention of cancer in an individual prone (genetically, due to life style, chronic inflammation and so forth) to develop cancer. As used herein, “prevention of cancer” is thus to be understood to include prevention of metastases, for example after surgical procedures or after chemotherapy.

According to some embodiments, the use further comprises administering an additional active agent such as an anti-cancer agent. The anti-cancer agent may be selected from anti-angiogenic agents, anti-proliferative agents and growth inhibitory agents. Thus, according to some embodiments, the pharmaceutical composition of the present invention is for use in combination with an additional active agent. The term “active agent” or “therapeutic agent” as used herein refers to a chemical entity or a biological product, or combination of chemical entities or biological products, which are used to treat, prevent or control a disease or a pathological condition.

According to some embodiments, the pharmaceutical composition is administered by any known method. According to one embodiment, the composition is administered via a route selected from parenteral, intravenous, arterial, intradermal, intramuscular, intraperitoneum, intravenous, subcutaneous, ocular, sublingual, oral (by ingestion), intranasal, via inhalation, and transdermal route of administration.

According to some embodiments, the pharmaceutical composition of the present invention is for use in inhibiting a PAR mediated signal transduction comprising administering a peptide or an analog thereof capable of selectively inhibiting binding of a G-protein coupled receptor (GPCR) comprising a Pleckstrin homology (PH) binding motif and a PH-domain containing protein, wherein said peptide is derived from a cytoplasmic tail (C-tail) of PAR4. According to one embodiment, the PAR mediated signal transduction is PAR4 mediated signal transduction. According to one embodiment, the PAR mediated signal transduction is PAR2 mediated signal transduction. According to one embodiment, the peptide is as described according to any one of the above aspects and embodiments. According to another embodiment, the analog, e.g. cyclic analog is as described according to any one of the above aspects and embodiments.

The term “PH-domain binding motif” refers to any structural motif capable of or configured to binging a PH-domain.

The terms “cytoplasmic tail”, “C-tail”, “cytoplasmic portion” or “cytoplasmic domain” are used herein interchangeably and refer to the C-terminus (carboxy-terminus) PH-domain binding motif of PAR. In one embodiment, C-tail is a C-tail of PAR4.

The term “PH-domain containing protein” refers to a protein which includes the pleckstrin homology (PH) domain. Such proteins are involved in signal transduction. Pleckstrin homology (PH) domain is a domain identified as a 100 to 120 amino acid stretch in more than 250 human proteins (Rebecchi, M. J. and Scarlata, S. Annu Rev Biophys Biomol Struct, 1998. 27: p. 503-28). Although the amino acid sequence of PH domains is not universally conserved, the tertiary structure is remarkably conserved. Non-limiting examples of PH-domain containing proteins are Etk/Bmx, Akt/PKB, Vav, SOS1 and GAB1.

According to another aspect, the present invention provides a method for inhibiting a G-protein coupled receptor (GPCR) mediated signal transduction comprising administering a peptide or an analog thereof capable of selectively inhibiting binding of the GPCR and a PH-domain containing protein, wherein said peptide is derived from a cytoplasmic tail (C-tail) of PAR4 and wherein the GPCR comprises a PH-domain binding motif.

According to another aspect, the present invention provides a method of treating a disease mediated by a protease-activated receptor (PAR) in a subject in need thereof comprising administering a peptide, peptide analog, a conjugate or a pharmaceutical composition comprising said peptide, analog or conjugate of the present invention. According to some embodiments, the disease is cancer. According to one embodiment, the method comprises killing cancer stem cells.

According to any one of the above aspects and embodiments, the GPCR is PAR. According to some embodiment, the PAR mediated signal transduction is mediated by PAR1, PAR2, PAR3 or PAR4 mediated signal transduction. According to some embodiments, the peptide or an analog are the peptide or the analog of the present invention.

According to some embodiments, the present invention provides a method of treating a disease in a subject in need thereof comprising administering a peptide or analog thereof or a conjugate thereof capable of selectively inhibiting binding of a GPCR comprising a PH-domain binding motif and a PH-domain containing protein, wherein said peptide is derived from a cytoplasmic tail (C-tail) of PAR4 and wherein the disease is mediated via binding of the GPCR and the PH-domain containing protein. According to some embodiments, the GPCR is PAR4 protein. According to another embodiment, the GPCR is PAR2 protein. According to yet another embodiment, the GPCR is selected from PAR4 and PAR2.

According to some embodiments, the disease mediated by PAR receptor is cancer. According to one embodiment, the present invention provides a method of treating cancer. According to some embodiments, treating cancer comprises killing cancer stem cells. According to some embodiments, the cancer is mediated via PAR4. According to some embodiments, the cancer is mediated via PAR2. According to any one of the below embodiments, the cancer expresses or overexpresses PAR4, PAR2 or both PAR4 and PAR2 proteins.

In some embodiments, the present invention provides a method of treating cancer selected from a cancer expressing ErbB protein and triple-negative breast cancer comprising administering a therapeutically effective amount of a peptide of the present invention as described in any one of the above aspects and embodiments, a salt, a cyclic analog of the peptide, or a conjugate thereof. In some embodiments, the cancer expressing ErbB protein is selected from HER2 positive (HER2+) cancer, epidermal growth factor receptor (EGFR) positive cancer, HER3 positive cancer and HER4 positive cancer. In some embodiments, the cancer overexpresses the ErbB protein.

In some embodiments, the present invention provides a method of treating cancer selected from caner expressing ErbB protein such as epidermal growth factor receptor (EGFR) positive cancer, breast HER2+ cancer, and ovarian HER2+ cancer, and triple-negative breast cancer, in a subject in need thereof, comprising administering a therapeutically effective amount of a peptide of the present invention as described in any one of the above or below aspects and embodiments, a salt, a cyclic analog of the peptide, or a conjugate thereof. According to any one of the embodiments, the cancer further expresses a GPCR selected from the group consisting of PAR4, PAR2 and both PAR4 and PAR2.

In some embodiments, the present invention provides a method of treating cancer in a subject in need thereof, comprising administering a therapeutically effective amount of a peptide comprising an amino acid sequence SZ1Z2FRDZ3 (SEQ ID NO: 2), a salt or a cyclic analog of the peptide, or a conjugate thereof, wherein said peptide consists of 7 to 25 amino acids, Z1 is an amino acid residue selected from alanine (Ala), a modified Ala, glycine (Gly), and a modified Gly; Z2 is a negatively charged amino acid; and Z3 is a positively charged amino acid, and wherein the cancer is selected from, wherein cancer overexpresses at least one GPCR selected from PAR4 and PAR2. In some embodiments, the cancer is selected from the group consisting of cancer expressing ErbB protein and triple-negative breast cancer. In some embodiments, the present invention provides a method of treating cancer selected from cancer expressing ErbB protein, HER2+ cancer, triple-negative breast cancer, ovarian HER2+ cancer, and triple-negative ovarian cancer in a subject in need thereof, comprising administering a therapeutically effective amount of a peptide comprising an amino acid sequence SZ1Z2FRDZ3, (SEQ ID NO: 1), a salt or a cyclic analog of the peptide, or a conjugate thereof, wherein Z1 is an amino acid selected from a hydrophobic amino acid, a modified hydrophobic amino acid, glycine, a modified glycine or histidine, Z2 is a negatively charged amino acid and Z3 is a positively charged amino acid, wherein said peptide consists of from 7 to 25 amino acids. In some embodiments, the method comprises treating triple-negative breast cancer. According to some embodiments, Z1 is an amino acid residue selected from alanine (Ala), a modified Ala, glycine (Gly), a modified Gly and histidine (His). According to some embodiments, the present invention provides a method of treating cancer in a subject in need thereof, comprising administering a therapeutically effective amount of a peptide comprising an amino acid sequence SZ1Z2FRDZ3, a salt or a cyclic analog of the peptide, or a conjugate thereof, wherein said peptide or analog consists of 7 to 25 amino acids, Z1 is an amino acid residue selected from alanine (Ala), a modified Ala, glycine (Gly), a modified Gly and histidine (His); Z2 is a negatively charged amino acid; and Z3 is a positively charged amino acid, and wherein the cancer is selected from breast HER2+ cancer, triple-negative breast cancer, ovarian HER2+ cancer, and triple-negative ovarian cancer. According to some embodiments, the cancer expressing ErbB is selected from the group consisting of EGFR positive cancer, HER2+ cancer, HER3+ and HER4+ cancerAccording to some embodiments, the peptide consists of 10 to 20 amino acids. According to another embodiment, the peptide consists of 10 to 15 amino acids. According to one embodiment, the peptide consists of 10, 11, 12, 13, 14 or 15 amino acids.

The term “triple-negative breast cancer” or “TNBC” as used herein refers to a type of breast cancer where no or little estrogen, progesterone and HER2 receptors are expressed by the tumor cells. Similarly, the term “triple-negative ovarian cancer” herein refers to a type of ovarian cancer where no or little estrogen, progesterone and HER2 receptors are expressed by the tumor cells

As used herein, the term “HER2+ breast cancer” and “HER2-enriched breast cancer” refers to breast cancers wherein at least a portion of the cancer cells express elevated levels of HER2 protein (HER2—human epidermal growth factor receptor 2 or HER2/neu) which promotes rapid growth of cells. Similarly, the terms “HER2+ ovarian cancer” and “HER2-enriched ovarian cancer” refer to ovarian cancers wherein at least a portion of the cancer cells express elevated levels of HER2 protein. According to some embodiments, the cancer comprises cancer expressing high levels of PAR4/f2r13. In some embodiments, the HER2+ cancer is selected from HER2+ breast cancer, HER2+ ovarian cancer, HER2+ bladder cancer, HER2+ ovarian pancreatic cancer, HER2+ ovarian gastric cancer and HER2+ colorectal cancer.

In some embodiments, the EGFR positive cancer is selected from lung adenocarcinoma, non-small cell lung carcinoma, conventional glioblastoma multiforme, glioblastoma, and colon adenocarcinoma.

In some embodiments, the HER3+ cancer is selected from HER3+ breast, ovarian, lung, colorectal, melanoma, head and neck, cervical and prostate cancer.

In some embodiments, the HER4+ cancer is selected from HER4+ colorectal cancer, gastric cancer, hepatocellular carcinoma, bladder cancer, lung cancer, breast cancer, ovarian cancer, pancreatic cancer, brain cancer, melanoma, endometrial cancer, and osteosarcoma.

According to some embodiments, the cancer is resistant to chemotherapy.

According to other embodiments, the present invention provides a method of treating cancer expressing epidermal growth factor receptor (EGFR, EGFR positive) and at least one GPCR selected from PAR2 and PAR4 in a subject in need thereof, comprising administering a therapeutically effective amount of a peptide or peptide analog of the present invention. According to some embodiments, the present invention provides a method of treating cancer expressing EGFR (EGFR positive) and at least one GPCR selected from PAR2 and PAR4 in a subject in need thereof, comprising administering a therapeutically effective amount of a peptide comprising an amino acid sequence SZ1Z2FRDZ3 (SEQ ID NO: 2), a salt or a cyclic analog of the peptide, or a conjugate thereof, wherein said peptide consists of 7 to 25 amino acids, Z1 is an amino acid residue selected from alanine (Ala), a modified Ala, glycine (Gly), and a modified Gly; Z2 is a negatively charged amino acid; and Z3 is a positively charged amino acid. According to some embodiments, the EGFR is selected from According to some embodiments, the EGFR positive cancer is a lung cancer.

According to some embodiments, the present invention provides a method of inhibiting activation of ErbB protein by at least one GPCR selected from PAR2 and PAR4 comprising administering a peptide of the present invention as described in any one of the above aspects and embodiments, a salt, a cyclic analog of the peptide, or a conjugate thereof. According to some embodiments, the ErbB protein is selected from EGFR and HER2. Thus, according to some embodiments, the present invention provides a method of inhibiting activation of EGFR by at least one GPCR selected from PAR2 and PAR4 comprising administering a peptide comprising an amino acid sequence SZ1Z2FRDZ3 (SEQ ID NO: 2), a salt or a cyclic analog of the peptide, or a conjugate thereof, wherein said peptide consists of 7 to 25 amino acids, Z1 is an amino acid residue selected from alanine (Ala), a modified Ala, glycine (Gly), and a modified Gly; Z2 is a negatively charged amino acid; and Z3 is a positively charged amino acid. Thus, according to some embodiments, the present invention provides a method of inhibiting activation of HER2 by at least one GPCR selected from PAR2 and PAR4 comprising administering a peptide comprising an amino acid sequence SZ1Z2FRDZ3 (SEQ ID NO: 2), a salt or a cyclic analog of the peptide, or a conjugate thereof, wherein said peptide consists of 7 to 25 amino acids, Z1 is an amino acid residue selected from alanine (Ala), a modified Ala, glycine (Gly), and a modified Gly; Z2 is a negatively charged amino acid; and Z3 is a positively charged amino acid.

Any above-described definitions and embodiments of the peptides, analogs, cyclic analogs and conjugates apply herein.

According to some embodiments, the method of treating cancer as defined hereinabove or inhibiting activation of ErbR protein such as EGFR comprises administering a peptide comprising an amino acid sequence SZ1Z2FRDZ3 (SEQ ID NO: 2), a salt or a cyclic analog thereof, wherein said peptide or analog consists of 7 to 25 amino acids, Z1 is an amino acid residue selected from alanine (Ala), a modified Ala, glycine (Gly), and a modified Gly; Z2 is a negatively charged amino acid; and Z3 is a positively charged amino acid. According to some embodiments, the peptide consists of 10 to 20 amino acids. According to another embodiment, the peptide consists of 10 to 15 amino acids. According to one embodiment, the peptide consists of 10, 11, 12, 13, 14 or 15 amino acids. According to some embodiments, Z2 is an amino acid selected from aspartic acid (Asp) and glutamic acid (Glu). According to other embodiments, Z3 is an amino acid selected from lysine (Lys), arginine (Arg) and His. According to yet another embodiment, Z2 is an amino acid selected from Asp and Glu, and, Z3 is an amino acid selected from Lys, Arg and His. According to one embodiment, Z2 is Glu and Z3 is Lys. Thus, according to some embodiments, the peptide comprises an amino acid sequence SZ1EFRDK (SEQ ID NO: 3) wherein Z1 is an amino acid residue selected from alanine (Ala), a modified Ala, glycine (Gly), and a modified Gly, a salt or an analog thereof wherein said peptide consists of 7 to 25 amino acids. According to some embodiments, the method comprises administering an analog of said peptide. According to some embodiments, Z1 is an amino acid selected from Ala, Val, Leu, Ile, Gly and His. According to some embodiments, Z1 is an amino acid selected from Ala and Gly (SEQ ID NO: 4). According to one embodiment, Z1 is Gly. According to another embodiment, Z1 is Ala. According to some embodiments, the peptide consists of from 8 to 20, 9 to 18, 10 to 16 or 12 to 16 amino acids. According to one embodiment, the peptide consists of 7, 8, 9, 10, 11, 12, 13, 14, 15, 16, 17, 18, 19, 20, 21, 22, 23, 24 or 25 amino acids. According to one embodiment, the peptide consists of 7 amino acids. According to another embodiment, the peptide consists of 12 amino acids.

According to some embodiments, the method comprises administering a peptide comprising the amino acid sequence SEQ ID NO: 2, wherein Z2 is an amino acid selected from aspartic acid (Asp) and glutamic acid (Glu). According to other embodiments, Z3 is an amino acid selected from lysine (Lys), arginine (Arg) and His. According to yet another embodiment, Z2 is an amino acid selected from aspartic acid (Asp) and glutamic acid (Glu) and Z3 is an amino acid selected from lysine (Lys), arginine (Arg) and His. According to some embodiments, the peptide comprises amino acid sequence X1X2SZ1EFRDKX3X4X5, wherein Z1 is an amino acid residue selected from Ala, Gly and His, X1 is a bulky hydrophobic amino acid such as Tyr, Phe, Ile and Trp, X2, X3 and X5 are each independently is a hydrophobic amino acid or Gly and X4 is a positively charged amino acid. According to other embodiments, the peptide comprises amino acid sequence X1X2SZ1EFRDKX3X4X5 (SEQ ID NO: 5), wherein Z1 is an amino acid residue selected from Ala, and Gly, X1 is a bulky hydrophobic amino acid such as Tyr, Phe, Ile and Trp, X2, X3 and X5 are each independently is a hydrophobic amino acid or Gly and X4 is a positively charged amino acid. According to one embodiment, wherein Z1 is Ala. According to another embodiment, Z1 is Gly. According to one embodiment, the hydrophobic amino acid is selected from Ala, Val, Leu, Ile, Gly, Phe and Trp. According to another embodiment, the positively changed amino acid is selected from Arg, Lys and His. According to some embodiments, the peptide consists of 7 to 25 amino acids. According to another embodiment, the peptide consists of 10 to 20 amino acids. According to yet another embodiment, the peptide consists of 10 to 15 amino acids. According to one embodiment, the peptide consists of 10, 11, 12, 13, 14 or 15 amino acids.

As used herein and in any one of the embodiments of the present invention, an amino acid denoted as Z is always present, and an amino acid denoted as X may be present or absent.

According to some embodiments, the method comprises administering a peptide comprising an amino acid sequences X1X2 SZ1EFRDKX3X4X5, wherein X1 is an amino acid selected from Tyr, Phe and Trp; X2, X3 and X5 are each independently an amino acid selected from Ala, Val, Leu, Ile, and Gly; X4 is an amino acid selected from Arg and Lys, and Z1 is an amino acid selected from Ala and Gly. According to one embodiment, Z1 is Ala. According to one embodiment, Z1 is Gly. According to one embodiment, the peptide comprises the amino acid sequence YVSAEFRDKVRA (SEQ ID NO: 6). According to another embodiment, the peptide comprises the amino acid sequence YVSGEFRDKVRA (SEQ ID NO: 7). According to some embodiments, the peptide consists of 7 to 25 amino acids. According to another embodiment, the peptide consists of 10 to 20 amino acids. According to yet another embodiment, the peptide consists of 10 to 15 amino acids. According to some embodiments, the peptide consists of the amino acid sequence YVSAEFRDKVRA. According to other embodiments, the peptide consists of the amino acid sequence YVSGEFRDKVRA.

According to one embodiment, the peptide of the present invention is capable of inhibiting activation of ErbB protein such as EGFR by PAR protein, e.g. by PAR4 or PAR2.

According to some embodiments, the method comprises administering an analog of the peptide described hereinabove.

According to any one of the above embodiments, the analog is a cyclic analog. Thus, according to some embodiments, the method of treating cancer of the present invention comprises administering a cyclic analog of a peptide according to any one of the above embodiments.

According to some embodiment, the method of the present invention comprises administering a cyclic analog comprising an amino acid sequence SZ (SEQ ID NO: 38), wherein Z1 is a hydrophobic amino acid, a modified hydrophobic amino acid, glycine, a modified glycine or histidine, Z2 is a negatively charged amino acid and Z3 is a positively charged amino acid, wherein said analog consists of from 7 to 25 amino acids. According to another embodiment, the cyclic analog consists of 10 to 15 amino acids. According to one embodiment, the cyclic analog consists of 10, 11, 12, 13, 14 or 15 amino acids. According to some embodiments, the hydrophobic amino acid is selected from Ala, Val, Leu, Ile, Gly, Phe, aminobutyric acid (Abu), Norvaline (Nva) and norleucine (Nle). According to some embodiments, the positively charged amino acid is selected from arginine, lysine, diaminoacetic acid, diaminobutyric acid, diaminopropionic acid, and ornithine. According to some embodiments, the negatively charged amino acid is selected from Asp, Glu, alpha-amino adipic acid (Aad), 2-aminoheptanediacid (2-aminopimelic acid) and alpha-aminosuberic acid (Asu). According to one embodiment, Z1 is a hydrophobic amino acid selected from Ala, Val, Leu, Ile, and Phe, or Gly or His. According to another embodiment, Z2 is a negatively charged amino acid selected from Asp, Glu, and aminoadipic acid, and Z3 is a positively charged amino acid selected from Lys, Arg and His, Dap, Dab and Orn. According to one embodiment, the cyclic analog comprises an amino acid sequence SZ1Z2FRDZ3, wherein Z1 is an amino acid selected from Ala, Val, Leu, Ile, Gly and His, Z2 is an amino acid selected from Asp and Glu, and Z3 is an amino acid selected from Lys, Arg and His, wherein said analog consists of from 7 to 25 amino acids. According to some embodiments, Z1 is Ala, Z2 is an amino acid selected from Asp and Glu, and Z3 is an amino acid selected from Lys and His. According to one embodiment, Z1 is Ala, Z2 is Asp, and Z3 is an amino acid selected from Lys and His. According to another embodiment, Z1 is Ala, Z2 is Glu, and Z3 is an amino acid selected from Lys and His. According to some embodiments, Z1 is Gly, Z2 is an amino acid selected from Asp and Glu, and Z3 is an amino acid selected from Lys and His. According to one embodiment, Z1 is Gly, Z2 is Asp, and Z3 is an amino acid selected from Lys and His. According to another embodiment, Z1 is Gly, Z2 is Glu, and Z3 is an amino acid selected from Lys and His. According to some embodiments, the cyclic analog consists of 10 to 20 amino acids. According to another embodiment, the cyclic analog consists of 10 to 15 amino acids. According to one embodiment, the cyclic analog consists of 10, 11, 12, 13, 14 or 15 amino acids. According to one embodiment, the cyclic analog comprises amino acid sequence SAEFRDK (SEQ ID NO: 8). According to another embodiment, the cyclic analog comprises amino acid sequence SADFRDH (SEQ ID NO: 9). According to a further embodiment, the cyclic analog comprises amino acid sequence SADFRDK (SEQ ID NO: 10). According to a certain embodiment, the cyclic analog comprises amino acid sequence SHDFRDH (SEQ ID NO: 11). According to another embodiment, the cyclic analog comprises amino acid sequence SHDFRDHA (SEQ ID NO: 37).

According to some embodiments, the cyclic analog comprises an amino acid sequence X1X2SZ1Z2FRDZ3X3X4X5, wherein Z1 is an amino acid selected from Ala, Val, Leu, Ile, Gly, a modified Ala, a modified Gly, and His, Z2 is a negatively charged amino acid and Z3 is a positively charged amino acid, X2, X3 and X5, if present, are each independently an amino acid selected from Ala, Val, Leu, Ile, Gly, a modified Ala, and a modified Gly, X1, if present, is an amino acid selected from Tyr, Phe and Trp and X4 if present is an amino acid selected from Arg and Lys, wherein said cyclic analog consists of from 7 to 25 amino acids. According to some embodiments, Z1 is His. According to some embodiments, wherein Z2 is an amino acid selected from Asp and Glu, and Z3 is selected from Lys, Arg and His. According to one embodiment, Z1 is His, Z2 is an amino acid selected from Asp and Glu, and Z3 is selected from Lys and His

According to some embodiments, the cyclic analog comprises an amino acid sequence X1X2SZ1Z2FRDZ3X3X4X5 (SEQ ID NO: 12), wherein Z1 is an amino acid selected from Ala, Val, Leu, Ile, Gly, a modified Ala, and a modified Gly, Z2 is a negatively charged amino acid and Z3 is a positively charged amino acid, X2, X3 and X5, if present, are each independently an amino acid selected from Ala, Val, Leu, Ile, Gly, a modified Ala, and a modified Gly, X1, if present, is an amino acid selected from Tyr, Phe and Trp and X4 if present is an amino acid selected from Arg and Lys, wherein said cyclic analog consists of from 7 to 25 amino acids. According to some embodiments, Z1 is selected from Ala, modified Ala, Gly and a modified Gly. According to some embodiments, the cyclic analog comprises an amino acid sequence SEQ ID NO: 12, wherein Z2 is an amino acid selected from Asp and Glu, and Z3 is selected from Lys, Arg and His. According to one embodiment, Z1 is an amino acid selected from Ala and Gly, Z2 is an amino acid selected from Asp and Glu, and Z3 is selected from Lys and His (SEQ ID NO: 36).

According to another embodiment, the cyclic analog comprises the amino acid sequence SEQ ID NO: 12, wherein Z1 is selected from Ala and Gly, Z3 is selected from Lys and His and X2 if present and X3 are each Val and X1, X4 and X5 are absent.

According to one embodiment, the cyclic analog comprises the amino acid sequence SEQ ID NO: 12 wherein Z1 is Gly, Z3 is selected from Lys and His, X3 is Gly, and X1, X2, X4 and X5 are absent. According to some embodiments, the cyclic analog comprises the amino acid sequence selected from VSGEFRDKG, SGEFRDKGV, VSGEFRDKGV, YVSGEFRDKG, YVSGEFRDKGV, SGEFRDKGVR, VSGEFRDKGVR, YVSGEFRDKGVR, SGEFRDKGVRA, VSGEFRDKGVRA, and YVSGEFRDKGVRA (SEQ ID NOs: 13-23).

According to some embodiments, the cyclic analog comprises an amino acid sequence SZ1Z2FRDZ3X3 (SEQ ID NO: 24), wherein Z1 and X3 are each independently an amino acid residue selected from Ala, a modified Ala, Gly, and a modified Gly, Z2 is an amino acid selected from Asp and Glu, and Z3 is an amino acid selected from Lys, Arg and His. According to some embodiments, Z1 is selected from Ala or Gly. According to other embodiments, Z2 is Glu. According to yet another embodiment, Z2 is Asp. According to certain embodiments, Z3 is Lys. According to other embodiments, Z3 is His. According to one embodiment, the cyclic analog comprises amino acid sequence SGEFRDKG (SEQ ID NO: 25). According to yet another embodiment, the cyclic analog comprises amino acid sequence SGDFRDHG (SEQ ID NO: 26). According to another embodiment, the cyclic analog comprises the amino acid sequence SGDFRDKG (SEQ ID NO: 27). According to yet another embodiment, the cyclic analog comprises the amino acid sequence SGEFRDHG (SEQ ID NO: 28). According to any one of the above embodiments, a pharmaceutically acceptable salt of said cyclic analog is contemplated.

According to some embodiment, the method of the present invention provides administering a cyclic analog comprising an amino acid sequence SEQ ID NO: 12, wherein, wherein Z1 and X3 are each independently a modified amino acid, Z2 is a negatively charged amino acid, Z3 is a positively charged amino acid and X1, X2, X4 and X5 are absent. According to some embodiments, the modified amino acids is selected from Na-w-functionalized and Ca-co-functionalized amino acid derivative. According to some embodiments, the modified amino acids are Na-w-functionalized amino acid derivatives (SEQ ID NO: 34). According to some embodiments, Z1 and X3 are each independently an amino acid selected from a modified Ala and a modified Gly. According to some embodiment, the present invention provides a cyclic analog comprising the sequence SZ1Z2FRDZ3X3 (SEQ ID NO: 35), wherein Z1 and X3 are each independently an amino acid selected from a modified Ala and a modified Gly, Z2 is selected from Asp and Glu and Z3 is selected from Lys and His. According to some embodiments, the modified amino acids are N^{α}-ω-functionalized amino acid derivatives. According to some embodiments, the Z1 and X3 are each independently selected from a Gly-BU and Ala-BU. According to another embodiment, the modified amino acids form a covalent bond is selected from an ester, amid, urea, thiourea, disulfide and guanoidino bond. Therefore, according to some embodiments, the cyclic analog is a backbone cyclic analog.

According to some embodiments, the method comprises administering a cyclic analog comprising the amino acid sequence SZ1Z2FRDZ3X3 (SEQ ID NO: 29), wherein Z1 and X3 are each independently selected from a Gly-BU and Ala-BU, Z2 is selected from Asp and Glu and, Z3 is selected from Lys and His. According to other embodiments, Z1 and X3 are each Ala-building unit. According to one embodiment, Z1 and X3 are each Gly-building unit. According to some embodiments, the Z1 and the X3 are covalently bound via an urea group. According to other embodiments, the Z1 and X3 are building units each individually comprising a (C1-C10) alkyl. According to another embodiment, the Z1 comprises 3, 4, 5 or 6 (CH)2 groups. According to another embodiment, X3 comprises 3, 4, 5 or 6 (CH)2 groups. According to some embodiments, the cyclic analog is a backbone cyclic analog. According to some embodiment, the cyclic analog comprises Z1 and X3, wherein the Z1 and X3 each individually a building unit comprising a (C3-C6) alkyl. According to further embodiment, the cyclic analog comprises Z1 and X3, wherein the Z1 and X3 each individually a building unit comprising a (C3-C5) alkyl. According to some embodiment, the cyclic analog comprises Z1 and X3, wherein the Z1 and X3 each individually a building unit comprising a (C3-C6) alkyl. According to one embodiment, Z1 comprises C3 alkyl and X3 comprises an alkyl selected from C3, C4, C5 and C6 alkyl. According to another embodiment, Z1 comprises C4 alkyl and X3 comprises an alkyl selected from C3, C4, C5 and C6 alkyl. According to yet another embodiment, Z1 comprises C5 alkyl and X3 comprises an alkyl selected from C3, C4, C5 and C6 alkyl. According to a further another embodiment, Z1 comprises C6 alkyl and X3 comprises an alkyl selected from C3, C4, C5 and C6 alkyl. According to some embodiments, the building unit are bound via a covalent bond to form backbone cyclization.

According to some embodiments, the method comprises administering a backbone cyclic analog comprising an amino acid sequence selected from SZ1EFRDKX3 (SEQ ID NO: 30) SZ1DFRDHX3 (SEQ ID NO: 31), SZ1EFRDHX3 (SEQ ID NO: 32), and SZ1DFRDKX3 (SEQ ID NO: 33), wherein Z1 and X3 are both Gly building units each comprising a (C2-C6) alkyl and are covalently bound via urea group. According to some embodiments, the Z1 and X3 are both Gly building units each comprising a (C3-05) alky covalently bound via urea group. According to other embodiments, the Z1 and X3 are both Gly building units each comprising a (C3-C6) alky covalently bound via urea group. According to some embodiments, the backbone cyclic analog consists of an amino acid sequence selected from SEQ ID NO: 30-33, wherein Z1 and X3 are both Gly building units each comprising a (C2-C6) alkyl and are covalently bound via urea group.

According to some embodiments, the method comprises administering a backbone cyclic analog comprising amino acid sequence SZ (SEQ ID NO: 30), wherein Z1 and X3 are both Gly building unit each comprising a (C3-C6) alky covalently bound via urea group. According to one embodiment, Z1 comprises C3 alkyl and X3 comprises an alkyl selected from C3, C4, C5 and C6 alkyl. According to another embodiment, Z1 comprises C4 alkyl and X3 comprises an alkyl selected from C3, C4, C5 and C6 alkyl. According to yet another embodiment, Z1 comprises C5 alkyl and X3 comprises an alkyl selected from C3, C4, C5 and C6 alkyl. According to a further another embodiment, Z1 comprises C6 alkyl and X3 comprises an alkyl selected from C3, C4, C5 and C6 alkyl. According to one embodiment, Z1 comprises C3 alkyl and X3 comprises C3 alkyl. According to one embodiment, Z1 comprises C3 alkyl and X3 comprises C4 alkyl. According to one embodiment, Z1 comprises C3 alkyl and X3 comprises C5 alkyl. According to one embodiment, Z1 comprises C4 alkyl and X3 comprises C3 alkyl. According to one embodiment, Z1 comprises C4 alkyl and X3 comprises C4 alkyl. According to one embodiment, Z1 comprises C4 alkyl and X3 comprises C5 alkyl. According to one embodiment, Z1 comprises C5 alkyl and X3 comprises C5 alkyl. According to some embodiments, the backbone cyclic analog has the structure of Formula I

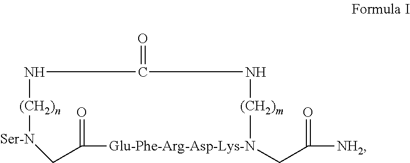

wherein n and m are each independently an integer between 3 and 6. According to some embodiments, n is 2 and m is selected from 3, 4, 5 and 6. According to other embodiments, n is 3 and m is selected from 2, 3, 4, 5 and 6. According to other embodiments, n is 4 and m is selected from 2, 3, 4, 5 and 6. According to further embodiments, n is 5 and m is selected from 2, 3, 4, 5 and 6. According to yet another embodiment, n is 6 and m is selected from 2, 3, 4 and 5. According to one embodiment, the peptidomimetic has the structure of Formula I, wherein m=n=4. In some embodiments of the invention the peptidomimetic having the structure of Formula I, wherein m=n=4 is referred also as PAR(4-4) and PAR 4×4 analog or inhibitor.

According to some embodiments, the method comprises administering a backbone cyclic analog comprising amino acid sequence SZ1DFRDHX3 (SEQ ID NO: 31), wherein Z1 and X3 are both Gly building unit each comprising a (C3-C6) alky covalently bound via urea unit. According to one embodiment, Z1 comprises C3 alkyl and X3 comprises an alkyl selected from C3, C4, C5 and C6 alkyl. According to another embodiment, Z1 comprises C4 alkyl and X3 comprises an alkyl selected from C3, C4, C5 and C6 alkyl. According to yet another embodiment, Z1 comprises C5 alkyl and X3 comprises an alkyl selected from C3, C4, C5 and C6 alkyl. According to a further another embodiment, Z1 comprises C6 alkyl and X3 comprises an alkyl selected from C3, C4, C5 and C6 alkyl. According to one embodiment, Z1 comprises C3 alkyl and X3 comprises C3 alkyl. According to one embodiment, Z1 comprises C3 alkyl and X3 comprises C4 alkyl. According to one embodiment, Z1 comprises C3 alkyl and X3 comprises C5 alkyl. According to another embodiment, Z1 comprises C4 alkyl and X3 comprises C3 alkyl. According to yet another embodiment, Z1 comprises C4 alkyl and X3 comprises C4 alkyl. According to a further embodiment, Z1 comprises C4 alkyl and X3 comprises C5 alkyl. According to one embodiment, Z1 comprises C5 alkyl and X3 comprises C5 alkyl.

According to some embodiments, the method comprises administering a backbone cyclic analog comprising an amino acid sequence selected from SZ1EFRDHX3 (SEQ ID NO: 32) and SZ1DFRDKX3 (SEQ ID NO: 33), wherein Z1 and X3 are both Gly building unit each comprising a (C3-C6) alky covalently bound via urea unit. According to one embodiment, Z1 comprises C3 alkyl and X3 comprises an alkyl selected from C3, C4, C5 and C6 alkyl. According to another embodiment, Z1 comprises C4 alkyl and X3 comprises an alkyl selected from C3, C4, C5 and C6 alkyl. According to yet another embodiment, Z1 comprises C5 alkyl and X3 comprises an alkyl selected from C3, C4, C5 and C6 alkyl. According to another embodiment, Z1 comprises C6 alkyl and X3 comprises an alkyl selected from C3, C4, C5 and C6 alkyl. According to one embodiment, Z1 comprises C3 alkyl and X3 comprises C3 alkyl. According to one embodiment, Z1 comprises C3 alkyl and X3 comprises C4 alkyl. According to one embodiment, Z1 comprises C3 alkyl and X3 comprises C5 alkyl. According to one embodiment, Z1 comprises C4 alkyl and X3 comprises C3 alkyl. According to one embodiment, Z1 comprises C4 alkyl and X3 comprises C4 alkyl. According to one embodiment, Z1 comprises C4 alkyl and X3 comprises C5 alkyl. According to one embodiment, Z1 comprises C5 alkyl and X3 comprises C5 alkyl.

According to some embodiment, the ring of the cyclic analog comprises from 20 to 50 atoms. According to other embodiments, the ring of the cyclic analog comprises from 22 to 48, from 25 to 45, from 28 to 43, from 30 to 40, from 32 to 38, or from 34 to 36 atoms. According to some embodiments, the ring of the cyclic analog comprises from 27 to 33 atoms, from 28 to 32 or from 39 to 31 atoms. According to some embodiments, the ring of the cyclic analog comprises 30 atoms. According to some embodiments, the ring of the cyclic analog comprises 29 atoms. According to some embodiments, the ring of the cyclic analog comprises 31 atoms. According to some embodiments, the ring of the cyclic analog comprises 28 atoms. According to some embodiments, the ring of the cyclic analog comprises 32 atoms. The term comprises has the meaning of consists of and may be replaced by it. Thus, according to some embodiments, the ring of the cyclic analog consists of from 20 to 50, from 22 to 48, from 25 to 45, from 28 to 43, from 30 to 40, from 32 to 38 or from 34 to 36 atoms, 28, 29, 30, 31 or 32 atoms.

According to some embodiments, the method comprises administering a pharmaceutically acceptable salt of said cyclic analog.

According to any one of the above embodiments, the methods of the present invention provide administering a conjugate of the peptide, peptide analog, cyclic peptide or cyclic analog as described hereinabove. According to one embodiment, the conjugate comprises a cyclic analog comprising an amino acid sequence selected from SEQ ID NO: 29-33. According to one embodiment, the conjugate comprises a cyclic analog consisting of an amino acid sequence selected from SEQ ID NO: 29-33. According to one embodiment, the conjugate comprises the cyclic analog having the structure of Formula I.

According to some embodiments, the method comprises administering a pharmaceutical composition comprising the peptide, cyclic peptide, analog or cyclic analog or the conjugate as described hereinabove.

According to another aspect, the present invention provides use of a peptide, peptide analog or a conjugate according to any one of the above aspects and embodiments, for preparation of a medicament for treating a disease mediated by protease-activated receptor (PAR). According to one embodiment, PAR is PAR4. According to some embodiments, the disease is cancer.

Having now generally described the invention, the same will be more readily understood through reference to the following examples, which are provided by way of illustration and are not intended to be limiting of the present invention.

### EXAMPLES

Material & Animal Models. Animals used in experiments were performed in accordance with the guidelines of the institution ethics committee (AAALAC standard). Mice were kept under SPF conditions at the Hadassah Medical Center animal facility unit of the Hebrew University and were regularly screened for standard pathogens. All animal experiments were approved by the animal committee of the Hebrew University (MD-20-15924-5).
Par4 mutant generation. PAR₄ C-tail mutation were constructed by replacing E to A (E/A), D to A D/A or F to L (F/L) using site-directed mutagenesis strategy. For additional methodology information see supplementary data.

### Example 1. PAR4 Harbors a PH-Binding Domain

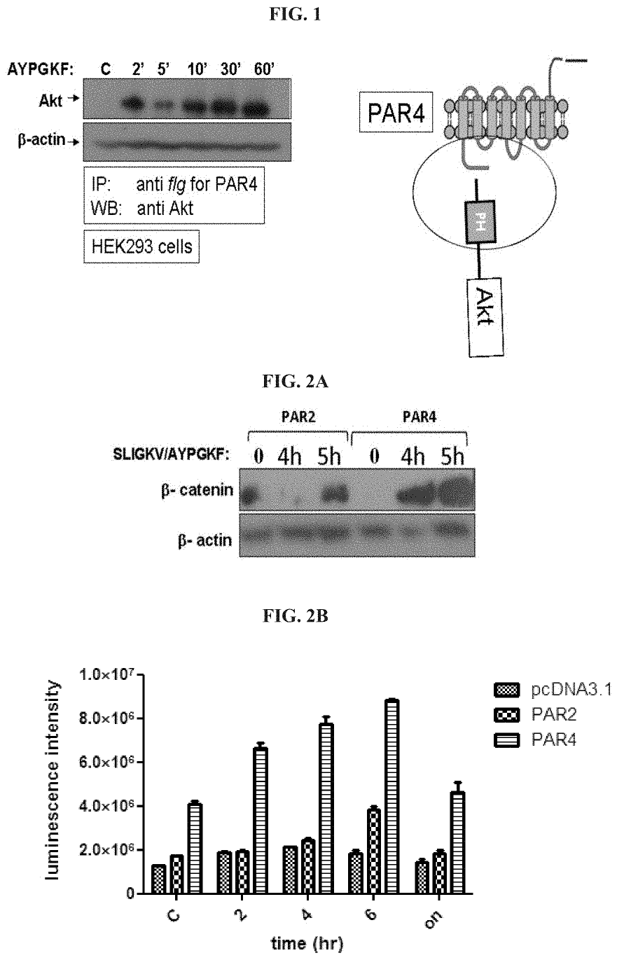

Nearly fibrocystic epithelial cells (HU cells) were transfected with flg-hPar4 plasmid. AYPGKF activation was carried out for up to 1 hour. Cell lysates were immunoprecipitated with anti-fig antibodies after defined periods of time, and Western blotted with anti Akt antibodies. As shown in FIG. 1, a potent complex formation between PAR4 and Akt, presumably via the PH-domain, was seen.

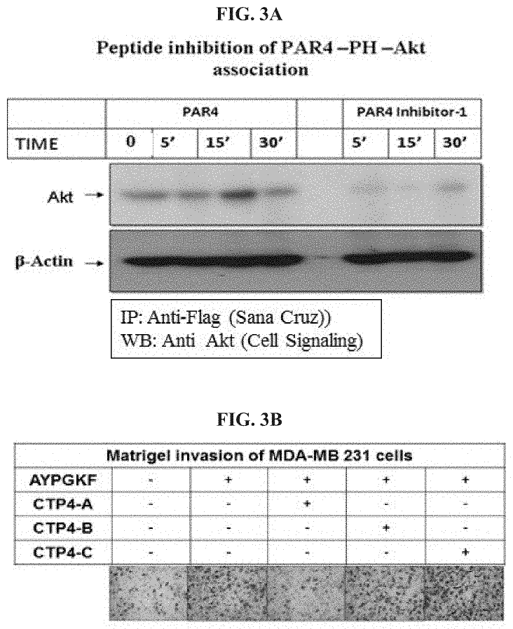

Further, it was shown that activation of PAR4 induced β-catenin level (FIG. 2A).

In addition TOPflash luciferase transcription activity was analyzed in HU cells following PAR4 activation in the presence of hPar4 wt construct. PAR4 activation elicits markedly elevated luciferase Lef/Tcf activity in HU cells as compared to PAR2 induced Lef/TCF levels (FIG. 2B). Maximal transcriptional activity was observed after 6 hrs activation. The results were evaluated using GraphPad InStat software and found to be statistically significant (p<0.01) and induces Lef/Tcf transcriptional activity.

These results clearly demonstrate that PAR4 plays a robust function in the stabilization of beta-catenin and consequently in a pathological molecular machinery that will lead to cancer.

### Example 2. Determination of Active Peptide

The entire C-tail PAR4 sequence is:

YVSAEFRDKVRAGLFQRSPGDTVASKASAEGGSRGMGTHSSLLQ.

Initially, we synthesized consecutive and overlapping (by two amino acid each) peptides of 12 amino acids each along the PAR4 C-tail. The following peptides were prepared:

Peptide 1:
H-YVSAEFRDKVRA-OH
Peptide 2:
H-RAGLFQRSPGDT-OH
Peptide 3:
H-DTVASKASAEGG-OH
Peptide 4:
H-GGSRGMGTHSSLLQ-OH (14 aa)

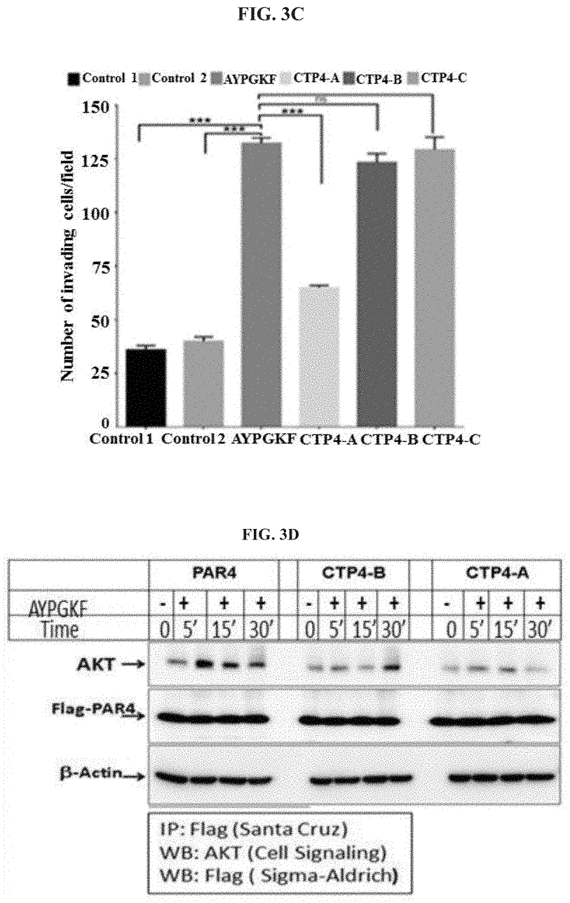

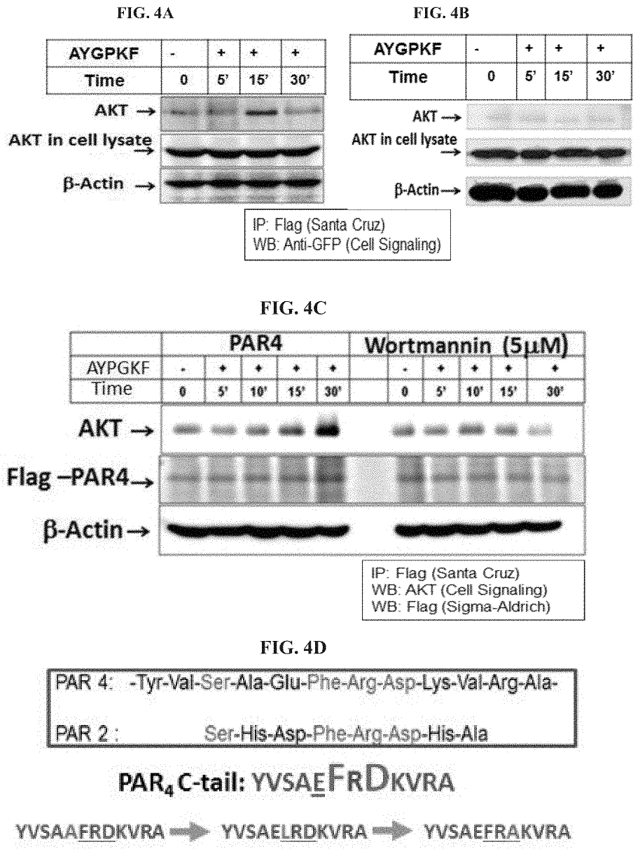

We evaluated the ability of these peptides to inhibit the binding of PAR4 to Akt protein. As can be seen from FIG. 3A, Peptide 1 (referred also as PAR4 Inhibitor-1) effectively inhibited interaction between PAR4 and Akt, which are assumable mediated by PH-domain or Akt.
In-addition, it is demonstrated that only peptide A (CTP4-A) is able to significantly inhibit PAR4 induced Matrigel invasion while no effect was observed by downstream peptides CTP4-B and CTP4-C, as shown in FIGS. 3B and 3C. Immunoprecipitation analyses further demonstrated that while PAR4 associates with AKT following AYPGKF activation, the association was inhibited in the presence of peptide A CTP4-A but not via CTP4-B (FIG. 3D).
Summarizing all said above, it is clear that Peptide 1; CTP4-A having the sequence YVSAEFRDKVRA effectively inhibited interactions between PAR4 and PH-domain of Akt protein.
The PH-domain of Akt/PKB associates with PAR4. To elucidate whether Akt/PKB binds via its PH-domain, constructs of either wt Akt/PKB PH-domain were applied alone or an Akt/PKB-PH domain mutant R25C, impaired in its lipid binding capability of the Akt/PKB-PH domain. Transient transfections of flg-hPar4 and either wt GFP-PH-domain or PH-domain R25C mutant were carried out in HEK293 cells. Cells were activated by the PAR4 AYPGKF ligand for the indicated periods of time and the lysates were further processed for IP analysis. Distinct binding of GFP-Akt/PKB-PH domain module alone with the PAR4 C-tail was obtained (FIG. 4A). In contrast, the R25C Akt-PH domain mutant, failed to associate with PAR4 (FIG. 4B), indicating the requirement of a lipid moiety for PAR4-Akt association. Along this line of evidence, the application of a PI3K inhibitor; Wortmannin, inhibited the otherwise potent association between PAR4 and Akt/PKB (FIG. 4C). Hence, the phosphatidylinositol (3,4,5)-trisphosphate [PtdIns(3,4,5)P3; PIP3] lipid is essential for this complex formation. It was concluded that the Akt/PKB-PAR4 binding association involves membrane lipid anchoring via the PAR4-PH-binding domain.
Critical roles for the PAR4 C-tail amino acids; F347 and D349 within the PH-binding motif. Analysis of both PH-binding domains of PAR2 and PAR4 reveals an identical core of three amino acids namely; Phe-Arg-Asp (FRD) which appears the same in both PH-binding motifs (FIG. 4D). This core sequence is critical for the proper association between PAR4 PH-binding motif and PH—signal proteins. Mutation inserted to the amino acid E346A outside the ‘FRD’ core sequence, did not affect the interaction of PAR4-Akt/PKB (FIG. 4E). In-contrast, mutations introduced within the ‘FRD’ core sequence effectively abrogated the association with PAR4-PH-Akt/PKB. For this purpose, mutant constructs of hPar4 F347L and hPar4 D349A were prepared and transiently transfected to HEK293 cells. The mutant constructs (e.g., wt: YVSAEFRDKVRA; mutant F347L: YVSAELRDKVRA=PAR4MutF; mutant D349A: YVSAEFRAKVRA=PAR4MutD) were further evaluated for association with Akt/PKB. While PAR4-Akt form a tight binding, neither mutant showed any association with Akt/PKB (FIG. 4F). It is concluded that either F or D (e.g., of the PH-binding motif FRD) are necessary and required for PAR4 and PH-Akt association.

### Example 3

### Preparation of Cyclic Peptide Analogs

Based on Peptide 1, a series of peptide analogs, and in particular backbone cyclic analogs were designed as described in Formula I and Table I:

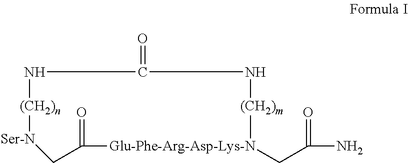

TABLE I
Design of cyclic analogs

| peptide # | n = | m = | ring size (atoms) |
|---|---|---|---|
| PAR(2-2) | 2 | 2 | 26 |
| PAR(2-3) | 2 | 3 | 27 |
| PAR(3-2) | 3 | 2 | 27 |
| PAR(3-3) | 3 | 3 | 28 |
| PAR(3-4) | 3 | 4 | 29 |
| PAR(3-6) | 3 | 6 | 31 |
| PAR(4-3) | 4 | 3 | 29 |
| PAR(4-4) | 4 | 4 | 30 |
| PAR(4-6) | 4 | 6 | 32 |
| PAR(6-3) | 6 | 3 | 31 |
| PAR(6-4) | 6 | 4 | 32 |
| PAR(6-6) | 6 | 6 | 34 |

These peptides are assessed by Matrigel invasion assay, in vitro. In order to evaluate the uptake of PAR₄ by epithelial cells and the half-life of the peptide analogs, the concentration of the donor compartment is quantified after 150 minutes of incubation in trans-wells coated with Caco-2, epithelial cells.

Several peptide analogs, specifically PAR(2-2), PAR(4-4) and PAR(6-6) were prepared.

### Example 4. Efficacy of PARc(4-4), PAR(2-2) and PAR(6-6) in Inhibiting Interactions of PAR₄ with AKT

Several peptide analogs according to Table 1 were prepared as described in Example 3 and tested for their ability to inhibit interactions with Akt protein.

HEK293 cells were transfected with 0.8 μg flag-hPar4 and serum starved over-night. The peptide analogs at concentration of 150 μM were applied onto the cell monolayer for 1 hr prior to PAR4 activation (by the peptide AYPGKF) for the indicated time periods (overall transfection period is 48 hrs). In additional experiments PARc(4-4) peptidomimetic at concentrations of 100, 75, 50 and 20 μM was tested.

Cells (HEK 293) were solubilized for 30 min at 40 C in lysis buffer containing 10 mM Tris-HCl, pH 7.4, 150 mM NaCl, 1 mM EDTA, 1% Triton X-100, a protease inhibitor cocktail (0.3 mM aprotinin, 1 mM PMSF; Sigma-Aldrich and 10 mM leupeptin). After centrifugation at 12,000 g for 20 min at 40 C, the supernatants were transferred and the protein content was measured.

Protein cell lysates (400 μg) were used for immunoprecipitation analysis. Anti-flag antibodies were added to the cell lysates. After overnight incubation, protein A-sepharose beads (Sigma-Aldrich) were added to the suspension, which was subsequently rotated at 40 C for 1 h. Elution of the reactive proteins was performed by resuspending the beads in protein sample buffer followed by boiling for 5 min. The supernatant was then resolved on a 10% SDS—polyacrylamide gel followed by transfer to Immobilon-P membrane (EMD Millipore/Merck, Damstadt, Germany). Membranes were blocked and probed with the appropriate antibodies. Anti-Akt antibody (Cell Signaling Technology and used at a dilution of 1:1,000). Anti b-actin was purchased from Sigma-Aldrich and used at a dilution of 1:5,000.

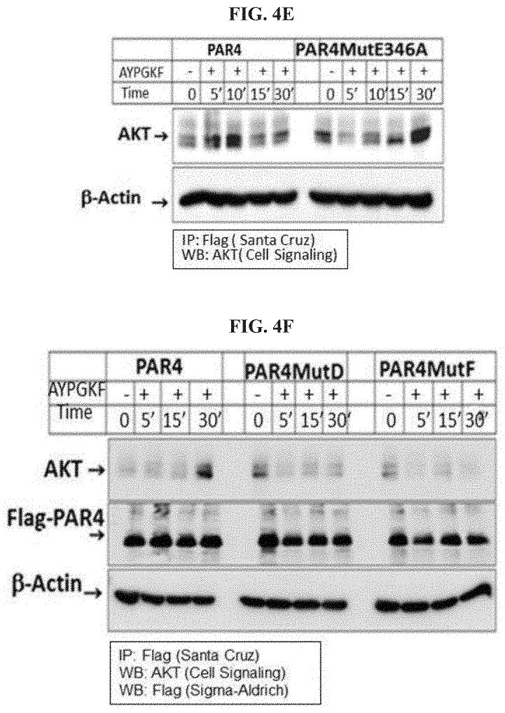

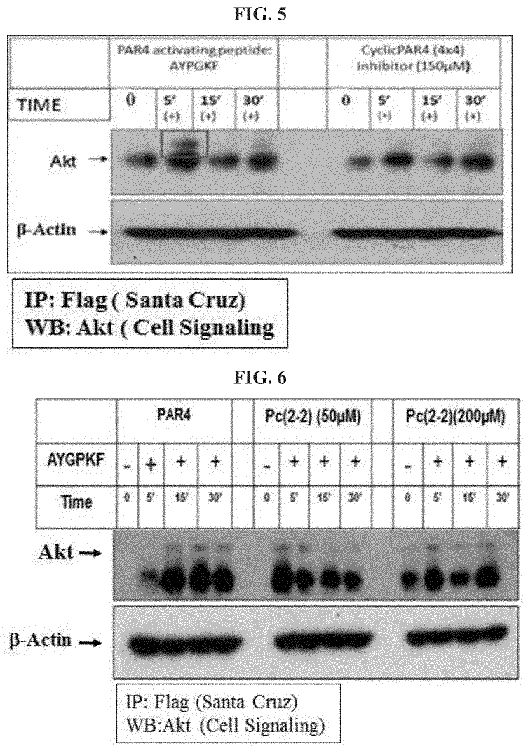

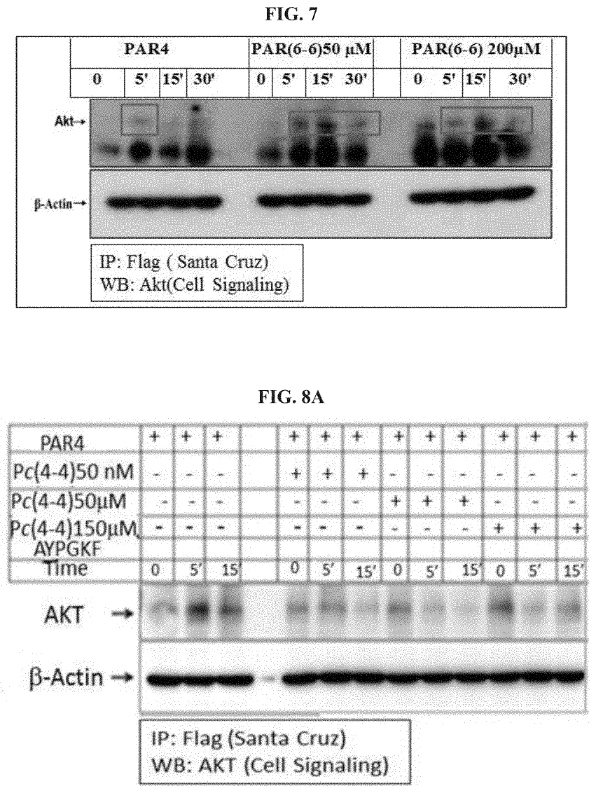

The results for PARc(4-4) are presented in FIG. 5, for PAR(2-2) in FIG. 6 and for PAR(6-6) in FIG. 7.

As can be seen from FIG. 6 and FIG. 7, both PAR(2-2) and PAR(6-6) were not effective in inhibiting PAR4-Akt association.

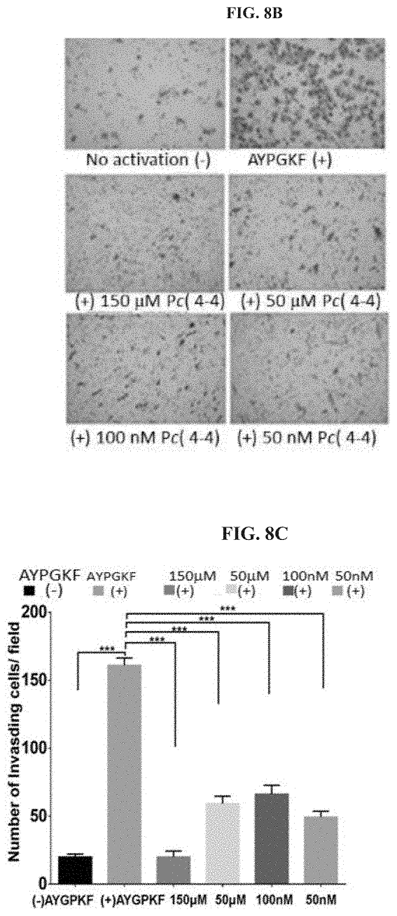

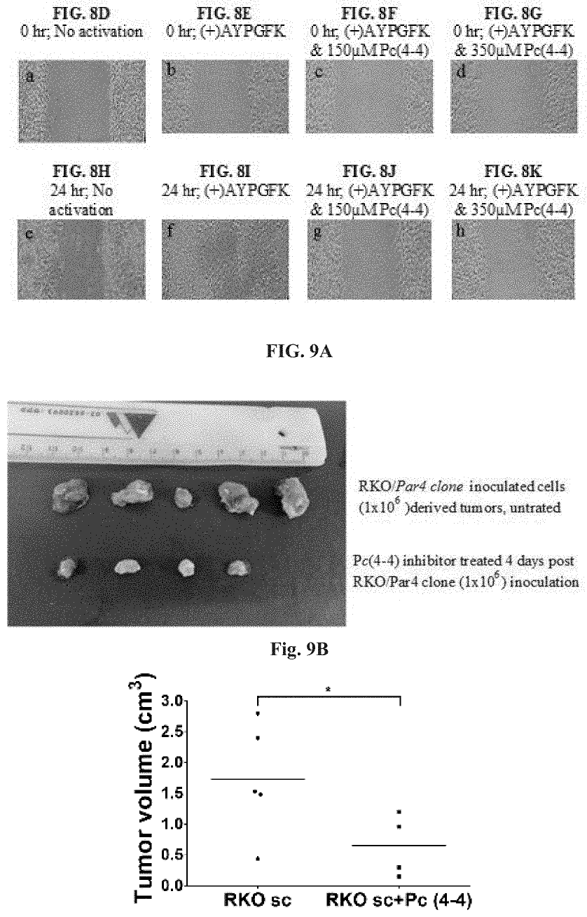

When we applied different concentrations of Pc(4-4) prior to the IP analysis, we observed a strong inhibition of Akt/PKB—PAR4 association between 50 nM-150 μM (FIG. 8A).

When we analyzed the impact of Pc(4-4) on the capability of activated PAR4 in Lovo colon cancer cell line to invade Matrigel, a robust invasion is seen (FIG. 8B), inhibited in the presence of Pc(4-4). This is observed within the range of 50 nM-150 μM (FIGS. 8B and 8C)

### Example 5. Efficacy of PAR(4-4) in Inhibiting Cell Proliferation and Migration

Migration was evaluated by “wound scratch assay” applied. An equal wide scratch was introduced to the monolayer cultures after overnight “cell starvation”. Application of the Pc(4-4) or not, were carried out on AYPGKF PAR4 activated monolayers. Consequently, after 24 hr the wound site is nearly filled up. In-contrast, in the presence of 150 μM and 300 μM of Pc(4-4), a marked inhibition of migration/proliferation is seen and the scratch remained nearly intact (FIG. 8D-8K).

### Example 6. Evaluation of Peptide Analogs

The most active peptide analogs are prepared in multi miligram quantity and subjected to the following pharmacological assays to determine their drug like properties: metabolic stability, intestinal permeability and pharmacokinetics (PK).

### Assessment of Intestinal Absorption Properties

Transport studies are be performed through the Caco-2 monolayer mounted in an Using-type chamber set-up with continuous transepithelial electrical resistance (TEER) measurements to assure TEER between 800 and 1200 Ω*cm². HBSS supplemented with 10 mM MES and adjusted to pH 6.5 will be used as transport medium in the donor compartment and pH 7.4 in the acceptor compartment. The donor solution will contain the test compound. The effective permeability coefficients will be calculated from concentration-time profiles of each of the tested compounds in the acceptor chamber.

### Pharmacokinetic (PK) Studies

The PK studies are be performed in conscious Wistar male rats. An indwelling cannula are be implanted in the jugular vein 24 hr before the PK experiment to allow full recovery of the animals from the surgical procedure. Animals (n=5) receive either an IV-bolus dose or oral dose of the investigated compound. Blood samples (with heparin, 15 U/ml) are be collected at several time points for up to 24 hrs post administration and re be assayed by HPLC-MS method. Noncompartmental pharmacokinetic analysis re be performed using WinNonlin software.

In order to evaluate the best PAR₄ cyclic peptidomimetic in vivo we analyze the ability of the selected peptidomimetic in a xenograft mouse model following inoculation of HCT116 cell line that highly express PAR₄. In-parallel we also inoculate stably expressing PAR₄ clone of RKO cells; RKO/Par4. The injection of the peptides analogs are carried out under 3 conditions:
Injection prior (3 days) to inoculation by HCT116 breast cancer cells or to RKO/Par4 clone stably expressing PAR₄;

Injection at the time of tumor cell inoculation;

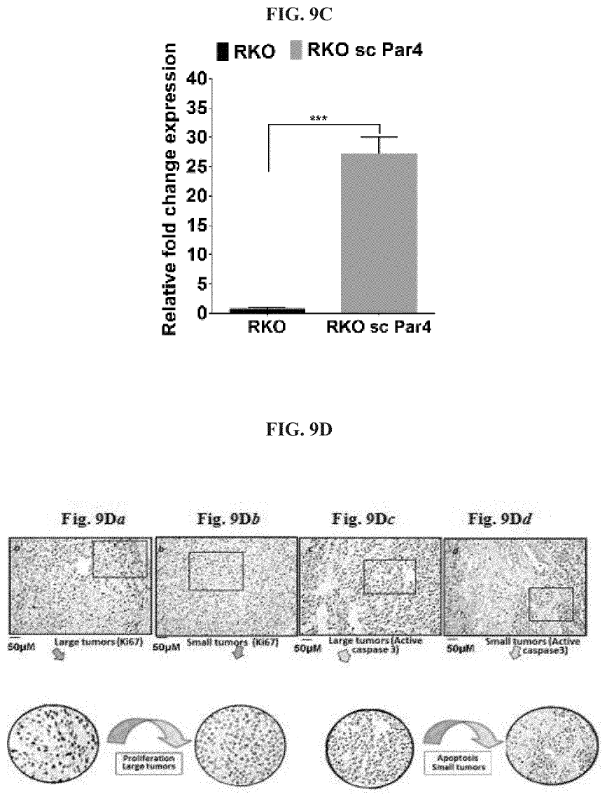

Injection 10 days after tumor cell inoculation. (either HCT116 or RKO/Par4 clone stably expressing PAR₄) subcutaneously (sc) (FIGS. 9A-9F&10A-10B).

### Example 7. In Vivo Evaluation of PARc (4-4) Peptide Analog on Cancer Growth

Methods

Having demonstrated the potent inhibitory effect of Pc(4-4), as preventing the association between Akt/PKB and PAR_{2&4}, consequently leading to inhibited migration and invasion in vitro, we next set out to evaluate its effect on tumor growth, in vivo. The physiological significance of the PAR₄ PH-binding motif in vivo is demonstrated by using a xenograft mouse model following s.c. inoculation of RKO/Par4 clone/s overexpressing PAR₄. Levels of PAR₄ in the generated RKO stable clones are shown (FIG. 9C).

### Preparation of Stable Clones

We have generated stable clones overexpressing PAR₄ in RKO cells, a non-aggressive colon cancer cell line (expressing wt p53), by infecting the cells with HA-hPar4 virus. This was followed by selection using Geneticin G418 resistance (500 μg/ml) to produce RKO/hPar4 clones. Clone efficiency generation was evaluated using qPCR analyses.

Another cell lines used in the experiment was HCT-116, an aggressive colon cancer cell line overexpressing oncogenes including PAR₄.

### Xenograft Tumor Mouse Model and Experiment Arrangement

Male athymic nude mice aged 6-7 weeks were pre-implanted subcutaneously with the relevant cells (1×10⁶ cells): either with HCT-116 or with RKO/hPar4 cells.

The inhibitor was injected at the site of the tumor at the day of inoculation. In an additional group, mice implanted with HCT-116 were administered with the inhibitor at day 4 after inoculation. The inhibitor (approximately 40 mg/kg) was applied repeatedly 3 times/week to all mice after the first administration of the inhibitor.

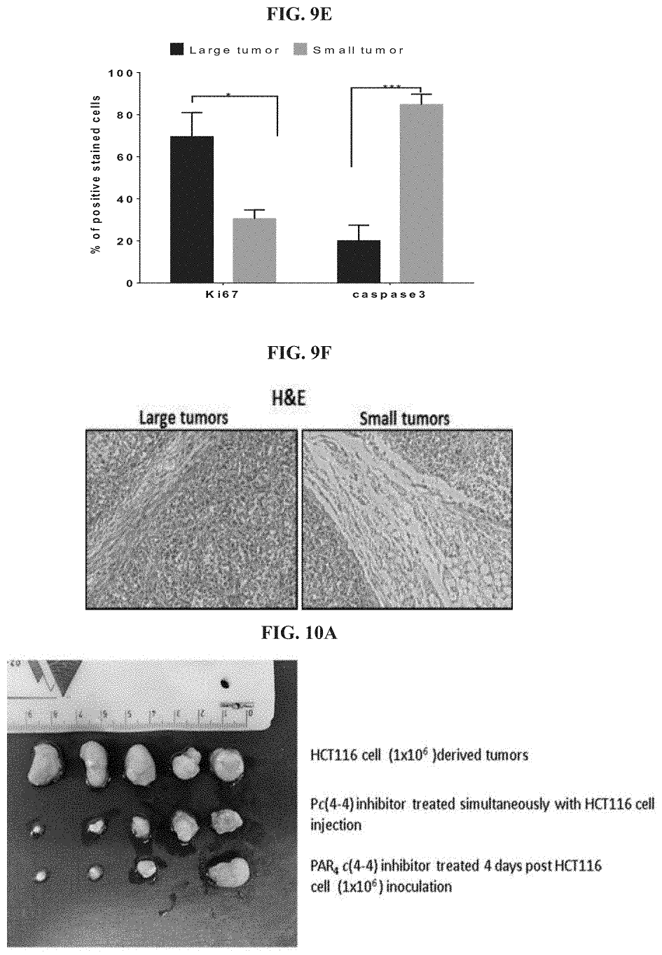

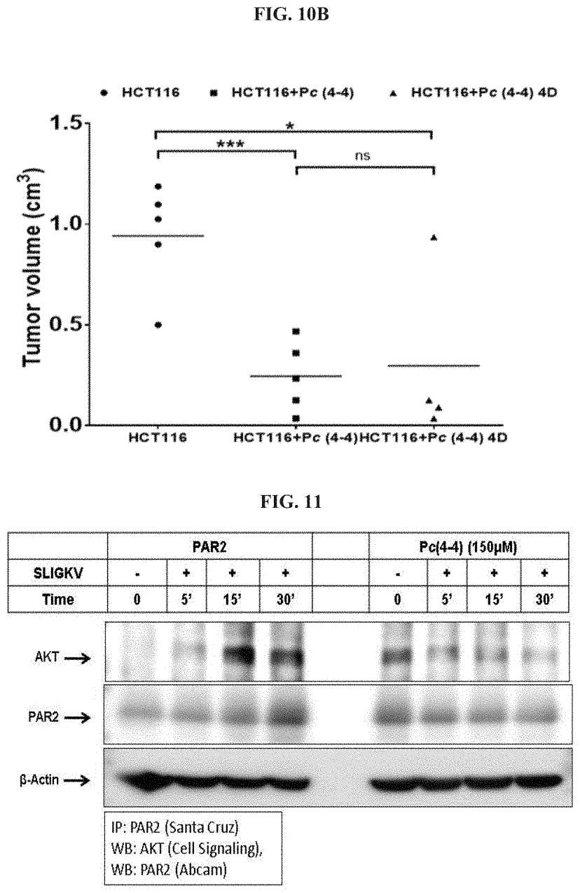

Mice were monitored for tumor size by external caliber measurements (length and width) on days 7, 14, 22 and for up to 32 days, if tumor burden allowed. Tumor volume (V) was calculated by V=L×W²×0.5, where L is length and W is width. At the end of the experiment, mice were euthanized and tumors were removed, weighed and fixed in formalin for histology. All mice survived to the end of the experiment. All animal experiments were approved by the animal committee of the Hebrew University (MD-19-15924). In fact, for the xenograft tumor formation we have utilized two types of cell lines; the HCT116 cells, an aggressive colon cancer cell line, overexpressing oncogenes among of which is also PAR₄, (FIGS. 10A and 10B) and clone/s overexpressing Par4, generated in RKO cell line; RKO/hPar4 cells (FIG. 9A-9C). HCT116 (1×10⁶) and RKO/hPar4 (1×10⁶) clone/s were inoculated subcutaneously in nude mice. The Pc(4-4) cyclic peptide was injected at the site of the tumor (100 μM; 0.25 mg/30 gram mouse), either at the time of tumor cell injection or 4 days after tumor-cell implantation. The Pc(4-4) cyclic peptide was applied repeatedly 3 times/week. The HCT116 cell inoculation generated large tumors and were terminated after 20 days, whereas the RKO/Par4 inoculation developed tumors on a slower pace and were terminated after 32 days. As can be seen, though large tumors were generated in both inoculated lines, significantly smaller tumors were observed in the presence of the Pc(4-4) (FIGS. 10A-10B and 9A-9B, respectively). The powerful inhibition of tumor growth was seen regardless of whether the Pc(4-4) cyclic peptide was applied at the same time of tumor inoculation or applied 4 days later. This was obtained irrespective of using an aggressive colon cancer cell line or stable RKO/hPar4 clone/s, overexpressing PAR₄ (FIGS. 9A and 9B).

IHC of large and small tumors have indicated increased proliferation in the large tumors as compared to little in the small tumors that were generated in the presence of Pc(4-4) as evaluated by ki67 staining (FIG. 9Da, 9Db, 9E). Accordingly, induced levels of active caspase-3, as a measure for apoptosis is seen in the small tumors while nearly none or very little in the large tumors obtained (FIG. 9Dc, 9Dd, 9E).

### Results

The HCT116 cell line inoculation generated large tumors and were terminated after 20 days, whereas the RKO/hPar4 inoculation developed tumors on a much slower paste and were terminated after 32 days. The results are presented in FIGS. 9A-9F and 10A-10B. As can be clearly seen from FIG. 9A-9F showing results obtained for mice inoculated with RKO/Par4 stable clones, markedly small tumors were observed in the presence of the PAR(4-4) (referred also as PAR₄ 4-4) inhibitor. There was no statistical difference between mice that started treatment at the day of inoculation or 4 days after inoculation. A statistical significance (T-test) was observed between the control (denoted as RKO/Par4 clone inoculated cells, and between control and mice treated from day 4 (Pc(4-4) inhibitor treated 4 days post RKO/Par4 clone (1×10⁶) inoculation).

Similarly, it can be clearly seen from FIGS. 10A and 10B that mice inoculated with HCT116 cells developed much smaller tumors in the presence of the PAR₄ (4-4) inhibitor than the untreated mice. This was obtained regardless of treatment with Pc (4-4) at the time of tumor cell (e.g., HCT116 cells) inoculation, or four days post-tumor inoculation (FIGS. 10A and 10B).

### Example 8. Efficacy of PAR(4-4) in Inhibiting Interactions of PAR₂ with AKT

In an experimental arrangement similar to that of Example 4, efficacy of PAR(4-4) cyclic peptidomimetic in inhibiting the interaction of PAR₂ with AKT was evaluated.

Briefly, HEK 293 cells were transfected with 0.8 μg flag-hPar2 and serum starved overnight. PAR(4-4) in concentration of 150 μM was applied onto the cell monolayer for 1 hr prior to PAR₂ activation (by the peptide SLIGKV—200 μM) for the indicated time periods (overall transfection period is 48 hrs).

The cells (HEK 293) were solubilized and lysed and protein cell lysates (400 μg) were used for immunoprecipitation analysis, as described in Example 4.

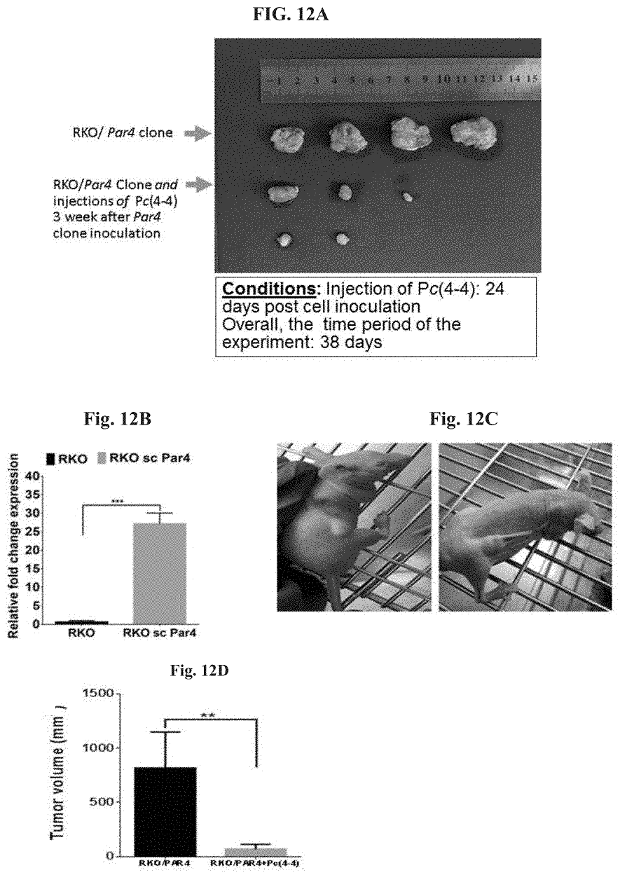

The results are presented in FIG. 11. As one can observe, after 5 min activation AKT was found in association with PAR₂ (left panel of FIG. 11). This association was efficiently inhibited in the presence of the cyclic PAR (4-4) inhibitor (right side panel of FIG. 11).

### Example 9

### Pc(4-4) Compound Inhibited Significantly Pre-Formed RKOIhPar4 Tumors

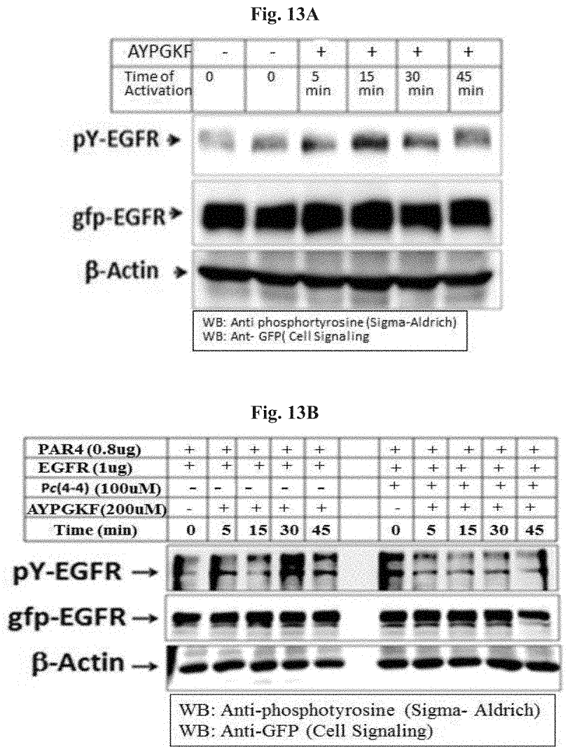

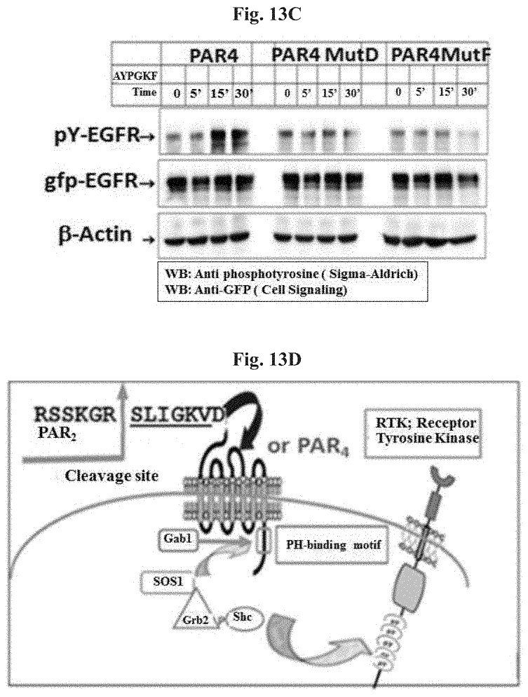

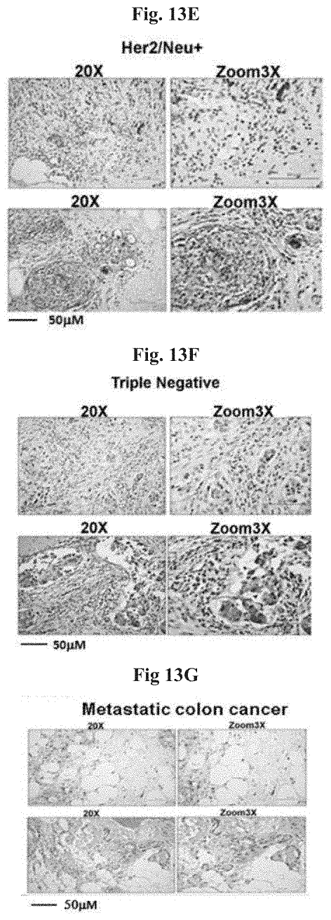

In this experimental design, RKO/hPar4 clone/s (1×10⁶ cells) were implanted in nude mice (Par4 levels in the clones) (FIG. 12A-12D). Tumors were slowly formed (during 3 weeks). At the time tumors were distinctly observed (˜1 cm length, FIG. 12C), the Pc(4-4) inhibitor was injected s.c. in the vicinity of the tumor site (3×week; 5 mg/kg). At the time the experiment was terminated large RKO/hPar4 tumors were observed. In contrast, distinctly smaller tumors were seen in the presence of Pc(4-4).
Activation of PAR₄ leads to the phosphorylation of EGFR.
AYPGKF activation of PAR₄ leads to pTyr-EGFR. This was shown by co-transfection of HEK293 cells with hPar4 and egfr followed by AYPGKF PAR₄ activation. Western blot detection of EGFR Tyr (Y)-phosphorylation (pY-EGFR), was carried-out by anti phospho-Tyr antibodies (FIG. 13A). The result indicated that activation of PAR₄ alone is sufficient to initiate activation of EGFR directly, as recapitulated by pY-EGFR. It may take place via the association of Sos1 with PAR4 PH-binding domain, linking Grbl and Shc for the direct association with the C-tail of EGFR. This describes an inside activation of EGFR via initiation of signaling events introduced by PAR4 activation. Indeed, when we applied or not Pc(4-4), following activation of PAR4 in HEK293 cells that were co-transfected with hPar4 and egfr, potent inhibition of the PAR4 induced pY-EGFR (FIG. 13B) was observed. Notably, PAR4 mutants, F347L and D349A failed to induce EGFR1 phosphorylation (FIG. 13C). FIG. 13D schematically depicts a plausible cross-talk between PAR and RTKs. To assess the clinical relevance of PAR2/4, we stained tissue from breast and colon cancer patients and found that it can be applicable also to Her2/Neu+ and TN patients that develop resistance to conventional therapy that express high levels of PAR₄/f2rl3 (FIGS. 13E and 13F) and EGFR/erbB. This, in addition to colon tumors expressing high levels of PAR₄/f2r/3 and PAR₂/f2rl1).
Kaplan-Meyer analysis of PAR expressing members in breast cancer. Combined analyses of Her2⁺ positive breast cancer patients with PAR₁/f2rl or PAR₂/f2rl1 and PAR₄/f2rl3 showed the following outcome. PAR members mRNA expression significantly correlated with a worse prognosis, expressed as overall survival (OS) time (FIG. 14A-14C). A significantly worse prognosis is seen in the presence of PAR₂ and PAR₄. These results underscore the significance of PAR family members especially PAR₂ and PAR₄, as aggressive genes impacting on the etiology of the disease. The proposed PAR₂/PAR₄ signaling in cancer is shown. The prospect for cancer intervention via novel PH-binding motif is presented.

### Expression of PAR₄ and EGFR in Breast Cancer MDA-MB-231 Cells

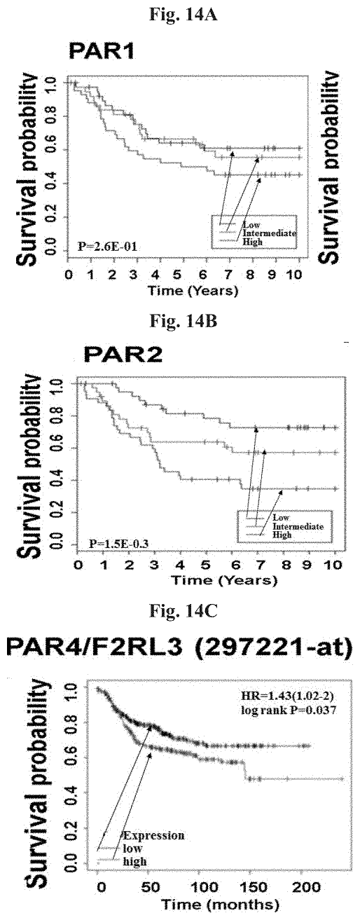

We analyzed the level of PAR₄ and EGFR receptors in MDA-MB-231 cells, an aggressive breast cancer cell line. As shown in FIG. 15, abundant expression levels of both PAR₄ and EGFR are seen in MDA-MB-231 cells.

### Immunohistochemistry of EGFR in Her2/Neu and TN Breast Cancer Patients Tissue

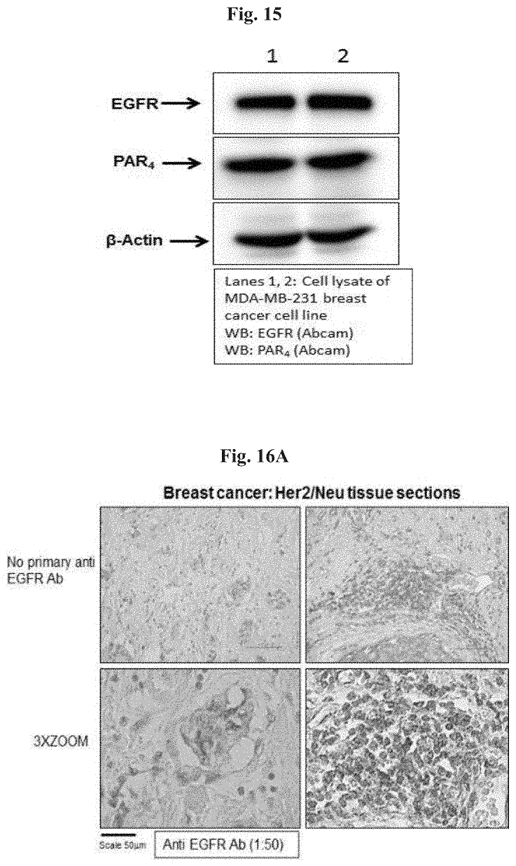

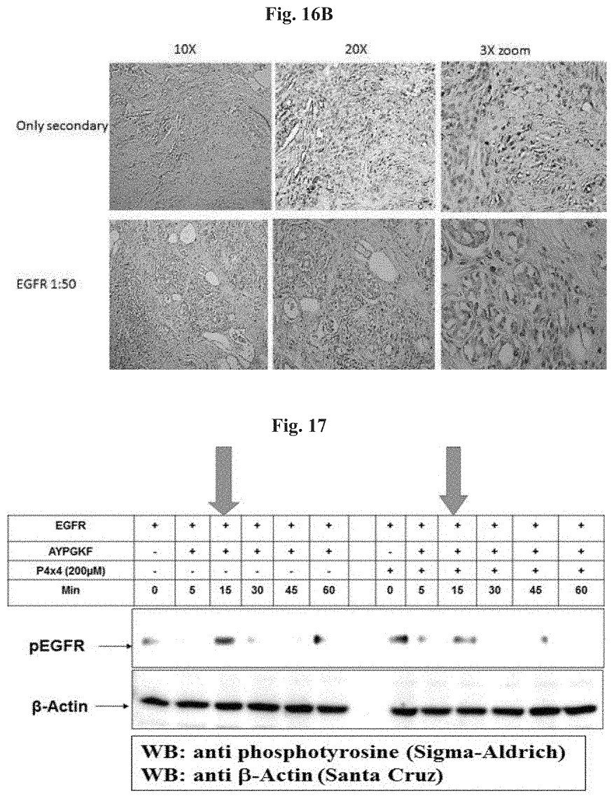

We have performed immunohistochemical (IHC) staining analyses of EGFR in Her2/Neu and TN breast cancer patient tissues. Analyses have indicated the presence of EGFR in both HER2/Neu as also in TN breast cancer tissue biopsy specimen (FIGS. 16A and 16B, respectively).
Pc(4-4) Inhibits PAR₄ Induced pEGFR in MDA-MB-231 Cells
MDA-MB-231 cells were serum starved overnight following AYPGKF PAR₄ activation (200 μM) for 5-60 min in the presence and absence of Pc(4-4) (200 μM). Detection by Western blot analyses of pEGFR and EGFR was performed using either anti-phosphotyrosine or anti-EGFR antibodies (1:1000 dilution), respectively. PAR₄ activation induces EGFR tyrosine phosphorylation, Pc(4-4), a PAR₄ inhibitor potently inhibited.
AYPGKF activation of PAR₄ leads to pTyr-EGFR in MDA-231 cells (see FIG. 17). This is demonstrated following 15 min activation of endogenous PAR₄. In the presence of Pc(4-4) cyclic peptide (right panel) potently (200 μM) inhibition of the phosphorylation of EGFR is observed (FIG. 17). Here, the effect of Pc(4-4) is demonstrated on endogenous EGFR and PAR₄, highly expressed in MDA-231, an aggressive cancer breast cell line.
Although the present invention has been described herein above by way of preferred embodiments thereof, it can be modified, without departing from the spirit and nature of the subject invention as defined in the appended claims.

1. A peptide comprising an amino acid sequence SZ₁Z₂FRDZ₃ (SEQ ID NO: 2), a salt or a cyclic analog thereof, wherein said peptide consists of 7 to 25 amino acids, Z₁ is an amino acid residue selected from alanine (Ala), a modified Ala, glycine (Gly), and a modified Gly; Z₂ is a negatively charged amino acid; and Z₃ is a positively charged amino acid.

2. The peptide of claim 1, wherein the peptide is characterized by:
(i) Z₂ is an amino acid selected from aspartic acid (Asp) and glutamic acid (Glu) and Z₃ is an amino acid selected from lysine (Lys), arginine (Arg) and His;
(ii) Z₁ is an amino acid residue selected from Ala and Gly, Z₂ is Glu and Z₃ is Lys, thereby the peptide comprises the amino acid sequence SZ₁EFRDK (SEQ ID NO: 4);
(iii) the peptide comprises an amino acid sequence X₁X₂SZ₁EFRDKX₃X₄X₅ (SEQ ID NO: 5), wherein X₁ is an amino acid selected from Tyr, Phe and Trp; X₂, X₃ and X₅ are each independently an amino acid selected from Ala, Val, Leu, Ile and Gly; and X₄ is an amino acid selected from Arg and Lys; or
(iv) the peptide comprises an amino acid sequence selected from YVSAEFRDKVRA (SEQ ID NO: 6) and YVSGEFRDKVRA (SEQ ID NO: 7).

3. A cyclic analog of the peptide according to claim 1.

4. The cyclic analog of claim 3, wherein the analog is characterized by at least one of (i) comprising the amino acid sequence SZ₁Z₂FRDZ₃ (SEQ ID NO: 1); and (ii) the ring size of the cyclic analog is from 29 to 35 atoms.

5. The cyclic analog of claim 4, comprising the amino acid sequence SZ₁Z₂FRDZ₃X₃ (SEQ ID NO: 24), wherein the analog is characterized by at least one of:
(i) Z₁ and X₃ are each independently an amino acid residue selected from Ala, a modified Ala, Gly and a modified Gly, Z₂ is an amino acid selected from Asp and Glu and Z₃ is an amino acid selected from Lys, Arg and His;
(ii) Z₁ is selected from Ala or Gly;
(iii) Z₂ is Glu; and
(iv) the analog comprises an amino acid sequence selected from SGEFRDKG (SEQ ID NO: 25) and SGDFRDHG (SEQ ID NO: 26).

6. The cyclic analog of claim 3, wherein the cyclic analog is a backbone cyclic analog.

7. The cyclic analog of claim 6, wherein the analog is characterized by at least one of:
(i) the analog comprises at least two non-contiguous modified amino acids capable of forming a covalent bond with each other to form a backbone cyclic analog;
(ii) the two modified amino acids are N^{α}-ω-functionalized amino acid derivatives capable of forming a covalent bond with another amino acid residue or with the a terminus of the peptide (building unit, BU);
(iii) each of the building units independently comprises a (C2-C6)alkyl; and
(iv) the covalent bond is selected from an ester, amid, urea, thiourea, disulfide and guanoidino bond.

8. The cyclic analog of claim 3, wherein the analog comprises an amino acid sequence SZ₁Z₂FRDZ₃X₃ (SEQ ID NO: 34), and the analog further characterized by at least one of:
(i) Z₁ and X₃ are each independently an N^{α}-ω-functionalized amino acid derivative building unit;
(ii) Z₁ and X₃ are selected from Gly-BU and Ala-BU; and
(iii) Z₁ and X₃ are covalently bound via urea group, thereby the cyclic analog is a backbone cyclic analog.

9. The cyclic analog of claim 8, wherein the analog is characterized by at least one of:
(i) Z₂ is selected from Asp and Glu and Z₃ is selected from Lys and His;
(ii) Z₁ and X₃ are both Gly building unit; and
(ii) Z₁ and X₃ are each independently comprising a (C3-C5)alkyl.

10. The cyclic analog of claim 9, wherein the analog comprises a sequence selected from SZ₁EFRDKX₃ (SEQ ID NO: 30) and SZ₁DFRDHX₃ (SEQ ID NO: 31), wherein Z₁ and X₃ are both Gly-BU units, each comprising a (C3-C6)alky covalently bound via urea group.

11. The cyclic analog of claim 10, wherein the cyclic analog has a structure of Formula I, 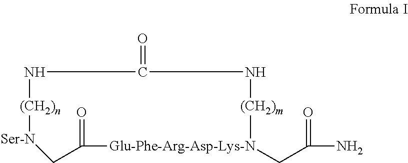
wherein n and m are each independently an integer between 3 and 6.

12. The cyclic analog of claim 11, wherein n=4 and m=4.

13. A conjugate of the peptide or the cyclic analog of claim 1.

14. A pharmaceutical composition comprising the peptide or the cyclic analog of claim 1 or the conjugate thereof, and a pharmaceutically acceptable excipient.

15. A method of treating a disease mediated by a protease-activated receptor (PAR) in a subject in need thereof comprising administering a peptide or cyclic analog of claim 1, the conjugate thereof, or a pharmaceutical composition comprising said peptide, analog or conjugate.

16. The method of claim 15, wherein the disease is cancer.

17. The method of claim 16, wherein the cancer is selected from the group consisting of cancer expressing ErbB protein, triple-negative breast cancer and a carcinoma.

18. The method of claim 17, wherein the cancer expressing ErbB is selected from the group consisting of EGFR-positive cancer, HER2+ cancer, HER3+ and HER4+ cancer.

19. The method of claim 18 wherein HER2+ cancer is selected from HER2+ breast cancer, HER2+ ovarian cancer, HER2+ bladder cancer, HER2+ ovarian pancreatic cancer, HER2+ ovarian gastric cancer and HER2+ colorectal cancer, and EGFR positive cancer is selected from lung adenocarcinoma, non-small cell lung carcinoma, glioblastoma, and colon adenocarcinoma.

20. A method for inhibiting G-protein coupled receptor (GPCR) mediated signal transduction comprising administering a peptide or a cyclic analog thereof or a conjugate thereof capable of selectively inhibiting binding of the GPCR and PH-domain containing protein, wherein said peptide is derived from a cytoplasmic tail (c-tail) of PAR₄ and the GPCR comprises a PH-domain binding motif.

21. A method of treating a disease in a subject in need thereof comprising administering a peptide or cyclic analog thereof or a conjugate thereof capable of selectively inhibiting binding of a GPCR comprising a PH-domain binding motif and a PH-domain containing protein, wherein said peptide is derived from a cytoplasmic tail (c-tail) of PAR₄, and wherein the disease is mediated via binding of the GPCR and the PH-domain containing protein.


SEQUENCE LISTING (38 sequences)

SEQ ID NO: 1 - protein, 7 aa, synthetic construct
  VARIANT 2: note = Xaa = a hydrophobic amino acid, a modified hydrophobic amino acid, glycine, a modified glycine or histidine
  VARIANT 3: note = Xaa = a negatively charged amino acid
  VARIANT 7: note = Xaa = positively charged amino acid
SXXFRDX

SEQ ID NO: 2 - protein, 7 aa, synthetic construct
  VARIANT 2: note = Xaa = Ala, modified Ala, Gly, or a modified Gly
  VARIANT 3: note = Xaa = negatively charged amino acid
  VARIANT 7: note = X-positively charged amino acid
SXXFRDX

SEQ ID NO: 3 - protein, 7 aa, synthetic construct
  VARIANT 2: note = Xaa = Ala, modified Ala, Gly, or a modified Gly
SXEFRDK

SEQ ID NO: 4 - protein, 7 aa, synthetic construct
  VARIANT 2: note = Xaa = Ala or Gly
SXEFRDK

SEQ ID NO: 5 - protein, 12 aa, synthetic construct
  VARIANT 1: note = Xaa = Ala or Gly
  VARIANT 2: note = Xaa = hydrophobic amino acid or Gly
  VARIANT 4: note = Xaa = Ala,or Gly
  VARIANT 10: note = Xaa = hydrophobic amino acid or Gly
  VARIANT 11: note = Xaa = positively charged amino acid
  VARIANT 12: note = Xaa = hydrophobic amino acid or Gly
XXSXEFRDKXXX

SEQ ID NO: 6 - protein, 12 aa, synthetic construct
  REGION 1..12: note = synthetic
YVSAEFRDKVRA

SEQ ID NO: 7 - protein, 12 aa, synthetic construct
  REGION 1..12: note = synthetic
YVSGEFRDKVRA

SEQ ID NO: 8 - protein, 7 aa, synthetic construct
  REGION 1..7: note = cyclic analog
SAEFRDK

SEQ ID NO: 9 - protein, 7 aa, synthetic construct
  REGION 1..7: note = cyclic analog
SADFRDH

SEQ ID NO: 10 - protein, 7 aa, synthetic construct
  REGION 1..7: note = cyclic analog
SADFRDK

SEQ ID NO: 11 - protein, 7 aa, synthetic construct
  REGION 1..7: note = cyclic analog
SHDFRDH

SEQ ID NO: 12 - protein, 12 aa, synthetic construct
  REGION 1..12: note = cyclic analog
  VARIANT 1: note = Xaa = Tyr, Phe, Trp or absent
  VARIANT 2: note = Xaa = Ala, Val, Leu, Ile, Gly, modified Ala, modified Gly or absent
  VARIANT 4: note = Xaa = Ala, Val, Leu, Ile, Gly, modified Ala, or modified Gly
  VARIANT 5: note = Xaa = negatively charged amino acid
  VARIANT 9: note = Xaa = positively charged amino acid
  VARIANT 10: note = Xaa = Ala, Val, Leu, Ile, Gly, modified Ala, modified Gly or absent
  VARIANT 11: note = Xaa = Arg, Lys or absent
  VARIANT 12: note = Xaa = Ala, Val, Leu, Ile, Gly, modified Ala, modified Gly or absent
XXSXXFRDXXXX

SEQ ID NO: 13 - protein, 9 aa, synthetic construct
  REGION 1..9: note = cyclic analog
VSGEFRDKG

SEQ ID NO: 14 - protein, 9 aa, synthetic construct
  REGION 1..9: note = cyclic analog
SGEFRDKGV

SEQ ID NO: 15 - protein, 10 aa, synthetic construct
  REGION 1..10: note = cyclic analog
VSGEFRDKGV

SEQ ID NO: 16 - protein, 10 aa, synthetic construct
  REGION 1..10: note = cyclic analog
YVSGEFRDKG

SEQ ID NO: 17 - protein, 11 aa, synthetic construct
  REGION 1..11: note = cyclic analog
YVSGEFRDKGV

SEQ ID NO: 18 - protein, 10 aa, synthetic construct
  REGION 1..10: note = cyclic analog
SGEFRDKGVR

SEQ ID NO: 19 - protein, 11 aa, synthetic construct
  REGION 1..11: note = cyclic analog
VSGEFRDKGVR

SEQ ID NO: 20 - protein, 12 aa, synthetic construct
  REGION 1..12: note = cyclic analog
YVSGEFRDKGVR

SEQ ID NO: 21 - protein, 11 aa, synthetic construct
  REGION 1..11: note = cyclic analog
SGEFRDKGVRA

SEQ ID NO: 22 - protein, 12 aa, synthetic construct
  REGION 1..12: note = cyclic analog
VSGEFRDKGVRA

SEQ ID NO: 23 - protein, 13 aa, synthetic construct
  REGION 1..13: note = cyclic analog
YVSGEFRDKGVRA

SEQ ID NO: 24 - protein, 8 aa, synthetic construct
  REGION 1..8: note = cyclic analog
  VARIANT 2: note = Xaa = Ala, modified Ala, Gly, or modified Gly
  VARIANT 3: note = Xaa = Asp or Glu
  VARIANT 7: note = Xaa = Lys, Arg or His
  VARIANT 8: note = Xaa = Ala, modified Ala, Gly, or modified Gly
SXXFRDXX

SEQ ID NO: 25 - protein, 8 aa, synthetic construct
  REGION 1..8: note = cyclic analog
SGEFRDKG

SEQ ID NO: 26 - protein, 8 aa, synthetic construct
  REGION 1..8: note = cyclic analog
SGDFRDHG

SEQ ID NO: 27 - protein, 8 aa, synthetic construct
  REGION 1..8: note = cyclic analog
SGDFRDKG

SEQ ID NO: 28 - protein, 8 aa, synthetic construct
  REGION 1..8: note = cyclic analog
SGDFRDHG

SEQ ID NO: 29 - protein, 8 aa, synthetic construct
  REGION 1..8: note = cyclic analog
  VARIANT 2: note = Xaa = N-alpha-omega-functionalized Ala or N-alpha-omega-functionalized Gly
  REGION 2..8: note = backbone cyclization
  VARIANT 3: note = Xaa = Asp or Glu
  VARIANT 7: note = Xaa = Lys or His
  VARIANT 8: note = Xaa = N-alpha-omega-functionalized Ala or N-alpha-omega-functionalized Gly
SXXFRDXX

SEQ ID NO: 30 - protein, 8 aa, synthetic construct
  REGION 1..8: note = backbone cyclic analog
  VARIANT 2: note = Xaa = N-alpha-omega-functionalized Gly comprising C2-C6 alkyl bound via urea group to Xaa at site 8
  REGION 2..8: note = backbone cyclization via urea group
  VARIANT 8: note = Xaa = N-alpha-omega-functionalized Gly comprising C2-C6 alkyl bound via urea group to Xaa at site 2
SXEFRDKX

SEQ ID NO: 31 - protein, 8 aa, synthetic construct
  REGION 1..8: note = backbone cyclic analog
  SITE 2: note = site - Xaa = N-alpha-omega-functionalized Gly comprising C2-C6 alkyl bound via urea group to Xaa at site 8
  REGION 2..8: note = backbone cyclization via urea group
  SITE 8: note = site - Xaa = N-alpha-omega-functionalized Gly comprising C2-C6 alkyl bound via urea group to Xaa at site 2
SXDFRDHX

SEQ ID NO: 32 - protein, 8 aa, synthetic construct
  REGION 1..8: note = backbone cyclic analog
  SITE 2: note = site - Xaa = N-alpha-omega-functionalized Gly comprising C2-C6 alkyl bound via urea group to Xaa at site 8
  REGION 2..8: note = backbone cyclization via urea group
  SITE 8: note = site - Xaa = N-alpha-omega-functionalized Gly comprising C2-C6 alkyl bound via urea group to Xaa at site 2
SXEFRDHX

SEQ ID NO: 33 - protein, 8 aa, synthetic construct
  REGION 1..8: note = backbone cyclic analog
  REGION 2..8: note = backbone cyclization via urea group
  SITE 2: note = N-alpha-omega-functionalized Gly comprising C2-C6 alkyl bound via urea group to Xaa at site 8
  SITE 8: note = Xaa = N-alpha-omega-functionalized Gly comprising C2-C6 alkyl bound via urea group to Xaa at site 2
SXDFRDKX

SEQ ID NO: 34 - protein, 8 aa, synthetic construct
  REGION 1..8: note = cyclic analog
  VARIANT 2: note = Xaa = N-alpha-omega-functionalized amino acid
  REGION 2..8: note = Backbone cyclization
  VARIANT 3: note = Xaa = negatively charged amino acid
  VARIANT 7: note = Xaa = positively charged amino acid
  VARIANT 8: note = Xaa = N-alpha-omega-functionalized amino acid
SXXFRDXX

SEQ ID NO: 35 - protein, 8 aa, synthetic construct
  REGION 1..8: note = cyclic analog
  VARIANT 2: note = Xaa = N-alpha-omega-functionalized Ala or N-alpha-omega-functionalized Gly
  REGION 2..8: note = backbone cyclization
  VARIANT 3: note = Xaa = Asp or Glu
  VARIANT 7: note = Xaa = Lys or His
  VARIANT 8: note = Xaa = Lys or His
SXXFRDXX

SEQ ID NO: 36 - protein, 12 aa, synthetic construct
  REGION 1..12: note = cyclic analog
  VARIANT 1: note = Xaa = Tyr, Phe, Trp or absent
  VARIANT 2: note = Xaa = Ala, Val, Leu, Ile, Gly, modified Ala, modified Gly or absent
  VARIANT 4: note = Xaa = Ala or Gly
  VARIANT 5: note = Xaa = Asp or Glu
  VARIANT 9: note = Xaa = Lys, Arg or His
  VARIANT 10: note = Xaa = Ala, Val, Leu, Ile, Gly, modified Ala, modified Gly or absent
  VARIANT 11: note = Xaa = Arg, Lys or absent
  VARIANT 12: note = Xaa = Ala, Val, Leu, Ile, Gly, modified Ala, modified Gly or absent
XXSXXFRDXXXX

SEQ ID NO: 37 - protein, 8 aa, synthetic construct
  REGION 1..8: note = cyclic analog
SHDFRDHA

SEQ ID NO: 38 - protein, 7 aa, synthetic construct
  REGION 1..7: note = cyclic analog
  VARIANT 2: note = Xaa = a hydrophobic amino acid, a modified hydrophobic amino acid, glycine, a modified glycine or histidine
  VARIANT 3: note = Xaa = a negatively charged amino acid
  VARIANT 7: note = Xaa = a positively charged amino acid
SXXFRDX